(19) United States
(12) Patent Application Publication
(10) Pub. No.: US 2007/0009497 A1
(43) Pub. Date: Jan. 11, 2007

(54) Dendritic cell expanded T suppressor cells and methods of use thereof

(76) Inventors: Ralph M. Steinman, Westport, CT (US); Kristin Tarbell, New York, NY (US); Sayuri Yamazaki, New York, NY (US)

Correspondence Address: PEARL COHEN ZEDEK, LLP;PEARL COHEN ZEDEK LATZER, LLP, 1500 BROADWAY 12TH FLOOR, NEW YORK, NY, 10036, US

(21) Appl. No.: 11/074,925

(22) Filed: Mar. 9, 2005

Related U.S. Application Data
(60) Provisional application No. 60/551,354, filed on Mar. 10, 2004.

(51) Int. Cl. A61K 48/00; A61K 39/395; C12N 5/08

(52) U.S. Cl. 424/93.21; 424/144.1; 435/372

(57) ABSTRACT

This invention relates to culture-expanded T suppressor cells and their use in modulating immune responses. This invention provides methods of producing culture-expanded T suppressor cells, which are antigen specific, and their use in modulating complex autoimmune diseases.

### CROSS-REFERENCE TO RELATED APPLICATIONS

This Application claims the benefit of U.S. Provisional Application Ser. No. 60/551,354, filed Mar. 10, 2004, which is hereby incorporated in its entirety.

### STATEMENT AS TO FEDERALLY SPONSORED RESEARCH

This invention was conducted with U.S. Government support under National Institutes of Health grant Number NIH 5 P01 AI 51573. The government has certain rights in the invention.

### FIELD OF THE INVENTION

This invention relates to culture-expanded T suppressor cells and their use in modulating immune responses. This invention provides methods of producing culture-expanded T suppressor cells, which are antigen specific, and their use in modulating complex autoimmune diseases.

### BACKGROUND OF THE INVENTION

Tolerance mechanisms for autoreactive T cells can be of “intrinsic” and “extrinsic” varieties. Intrinsic mechanisms include deletion and anergy of self-reactive T cells, while extrinsic mechanisms include different regulatory T cells that suppress other self-reactive T cells. One type of extrinsic suppressor is the CD25⁺ CD4⁺ T cell, which constitutes 5-10% of CD4⁺ peripheral T cells. These are produced in the thymus and maintain tolerance to self-antigens, as well as play a role in other immune responses, such as in infection, transplants and graft versus host disease.

The transcription factor, FoxP3, is important for CD25⁺ CD4⁺ T cell suppressor activity, and children who are born with defective FoxP3 rapidly develop autoimmunity, such as, for example, autoimmune diabetes. Models for the study of autoimmunity have played a critical role in both the understanding of the pathogenesis, and the devising of therapeutic strategies for these diseases. In a mouse model of autoimmune diabetes, the non-obese diabetic (NOD) mice, for example, CD25⁺ CD4⁺ regulatory T cells inhibit diabetes development, making this extrinsic tolerance mechanism an attractive target to develop antigen-specific therapies for autoimmune disease. In an experimental model of multiple sclerosis mediated by transgenic T cells specific to myelin basic protein, CD25⁺ CD4⁺ T cells specific for this antigen showed better suppression of disease than CD25⁺ CD4⁺ T cells with TCRs specific for other antigens. These findings suggest a role for antigen-specific CD25⁺ CD4⁺T cells, in suppressing autoimmunity, though it remains unclear whether CD25⁺ CD4⁺ T cells of one antigen specificity, can suppress autoimmune disease, caused by T cell responses to many autoantigens.

In vitro, CD25⁺ CD4⁺ T cells will suppress the proliferative or cytokine responses of naive CD25⁻ CD4⁺ T cells, however, the CD25⁺ CD4⁺ T cells are themselves unable to proliferate, are anergized, when stimulated by antigen presenting cells (APCs), in vitro. It is therefore unclear how the numbers of regulatory T cells are sustained and expanded, in vivo. Further, CD25⁺ CD4⁺ T cell expansion in vitro is as yet limited, further confounding their application in therapeutic settings

### SUMMARY OF THE INVENTION

This invention provides, in one embodiment, an isolated, culture-expanded T suppressor cell population, wherein the population expresses CD25 and CD4 on its cell surface. In one embodiment, the culture-expanded T suppressor cell population is antigen specific. In one embodiment, the culture-expanded T suppressor cell population expresses a monoclonal T cell receptor, or in another embodiment, expresses polyclonal T cell receptors.

In one embodiment, this invention provides a method for producing an isolated, culture-expanded T suppressor cell population, comprising contacting CD25+ CD4+ T cells with dendritic cells and an antigenic peptide, an antigenic protein or a derivative thereof, or an agent that cross-links a T cell receptor on said T cells in a culture, for a period of time resulting in antigen-specific CD25+ CD4+ T cell expansion and isolating the expanded CD25+ CD4+ T cells thus obtained, thereby producing an isolated, culture-expanded T suppressor cell population. In one embodiment, the method further comprises the step of adding a cytokine to the dendritic cell, CD25+ CD4+ T cell culture, which in one embodiment, is interleukin-2. In one embodiment, the dendritic cells are selected for their capacity to expand antigen-specific CD25+CD4+ suppressor cells.

According to this aspect of the invention, and in one embodiment, the dendritic cells are isolated from a subject suffering from an autoimmune disease or disorder, and in another embodiment, the antigenic peptide or antigenic protein is associated with the autoimmune disease or disorder. In one embodiment, the dendritic cells are isolated from a subject with an inappropriate or undesirable inflammatory response, and in another embodiment, the antigenic peptide or protein is associated with the inappropriate or undesirable inflammatory response. In one embodiment, the dendritic cells are isolated from a subject with an allergic response, and in another embodiment, the antigenic peptide or protein is associated with the allergic response. In one embodiment, the dendritic cells are isolated from a subject who is a recipient of a transplant, or in another embodiment, from a donor providing a transplant to said subject. In one embodiment, according to this aspect of the invention, the antigenic peptide or protein is associated with an immune response in the subject receiving a transplant from a donor. In one embodiment, the immune response is a result of graft versus host disease, or in another embodiment, the immune response is a result of host versus graft disease.

In one embodiment, this invention provides a method for delaying onset, reducing incidence or suppressing an autoimmune response in a subject, comprising the steps of contacting in a culture CD25+ CD4+ T cells with dendritic cells and an antigenic peptide or an antigenic protein associated with an autoimmune response in a subject, for a period of time resulting in CD25+ CD4+ T cell expansion; and administering the expanded CD25+ CD4+ T cells thus obtained to a subject, wherein the isolated, expanded CD25+ CD4+ T cells suppress an autoimmune response in the subject, thereby delaying onset, reducing incidence or otherwise suppressing an autoimmune response.

In one embodiment, this invention provides a method for downmodulating an immune response in a subject, comprising the steps of contacting in a culture CD25+ CD4+ T cells with dendritic cells and an antigenic peptide or an antigenic protein associated with an immune response in a subject, for a period of time resulting in CD25+ CD4+ T cell expansion; and administering the expanded CD25+ CD4+ T cells thus obtained to a subject, wherein said isolated, expanded CD25+ CD4+ T cells down modulate an immune response in said subject. In one embodiment one or more specificities, including a mixture of antigens derived from a (for diabetes) pancreatic beta cell line or islet tissue itself.

In one embodiment, this invention provides a method for delaying onset, reducing incidence or suppressing an autoimmune response in a subject, comprising the steps of culturing an isolated dendritic cell population with an antigenic peptide or an antigenic protein associated with an autoimmune response in a subject and administering the dendritic cells to a subject, whereby the dendritic cells contact CD25+ CD4+ T cells, resulting in CD25+ CD4+ T cell expansion in the subject, wherein expanded CD25+ CD4+ T cells suppress an autoimmune response in the subject, thereby delaying onset, reducing incidence or suppressing an autoimmune response. In one embodiment one or more specificities, including a mixture of antigens derived from a (for diabetes) pancreatic beta cell line or islet tissue itself.

In one embodiment, this invention provides a method for downmodulating an immune response in a subject, comprising the steps of culturing an isolated dendritic cell population with an antigenic peptide or an antigenic protein associated with an immune response in a subject and administering the dendritic cells to a subject, whereby the dendritic cells contact CD25+ CD4+ T cells, resulting in CD25+ CD4+0 T cell expansion in the subject, wherein expanded CD25+ CD4+ T cells downmodulate an immune response in the subject.

In one embodiment, this invention provides a method for delaying onset, reducing incidence or suppressing an autoimmune response in a subject, comprising the step of contacting a dendritic cell population in vivo with an antigenic peptide or protein associated with an autoimmune response in the subject for a period of time whereby the dendritic cells contact CD25+ CD4+ T cells in the subject, stimulating antigen-specific expansion of the CD25+ CD4+ T cells in the subject, wherein expanded CD25+ CD4+ T cells suppress an autoimmune response in the subject, thereby delaying onset, reducing incidence or otherwise suppressing an autoimmune response.

In another embodiment, this invention provides a method for modulating an immune response in a subject, comprising the steps of contacting a dendritic cell population in vivo with an antigenic peptide or protein associated with an immune response whose modulation is desired, whereby the dendritic cell population contacts CD25+ CD4+ T cells in the subject, wherein CD25+ CD4+ T cell contact promotes antigen persistence in said dendritic cell population in vivo, and the dendritic cell population with persistent antigen contacts effector T cells in the subject, wherein the effector T cells modulate an immune response associated with the antigenic protein or peptide thereby modulating an immune response in a subject.

### BRIEF DESCRIPTION OF THE DRAWINGS

FIG. 1 demonstrates DCs stimulate CD25+ CD4+ T cell growth. (A) CD25+ or CD25− CD4+ FACS-purified (top), DO11.10, OVA-specific T cells (1×104) were cultured 3d with spleen APCs (10⁵) or CD86+ mature DCs (5×10³) and anti-CD3 mAb, and 3H-thymidine uptake assessed (60-72 h). (B) As in (A), but T cells were from two OVA specific TCR transgenic mice, DO11.10 and OT-2, and the DCs were pulsed or not pulsed with 1 mg/ml OVA protein. (C) CD25+ CD4+ T cells from wild type BALB/C mice (closed diamonds) proliferate in response to DCs presenting anti-CD3 (right) but not OVA (left). (D, E) Day 6 marrow DCs were FACS separated into mature CD86high and immature CD86low CD11c+ subsets (D) and cultured with CD25+ CD4+ DO11.10 T cells (E) with OVA protein (1 mg/ml pulsed onto the DCs) or OVA 323-339 peptide (1 μg/ml) continuously. One representative result of at least three experiments is shown.

FIG. 2 demonstrates A large fraction of CD25+ CD4+ T cells are driven into multiple cell cycles by DCs. (A) As in FIG. 1, but the kinetics of proliferation (3H-thymidine and cell counts) were both followed. (B) CFSE labeled, T cells (1×104) were cultured 3d with 104 CD86+ mature BMDCs either OVA-pulsed (DC-OVA) or unpulsed (DC), prior to FACS analysis. (C) Quantitative estimation of the number of T cells entering cell cycle, and the number of mitotic events, was carried out as follows. CFSE-labeled CD25+ CD4+ T cells T cells (1×104) were cultured for 72 h with 1 mg/ml OVA pulsed CD86+ BMDCs (104), and analyzed for dilution of CFSE label (C). The percentage of total CD4+ events under each division peak (a) was experimentally determined (b). In this experiment, 24,000 live T cells were recovered, from which the absolute T cell count in each division peak at the time of harvest could be calculated (c). The absolute number of original, or precursor, T cells required to have generated these daughters is extrapolated by dividing the numbers of cells in column “c” by the number of divisions, 2n (d). The sum of the number of precursors giving rise to each peak represents the number of T cells at day 0 that entered cell cycle, which in this experiment was 3834 (the sum of column (d)) from a starting number of 10,000 T cells, giving a precursor frequency of 38%. The number of progeny in each peak (c) minus the number of precursors giving rise to the progeny (d) gives the number of mitotic events (e). The sum of these events represents the total number of cell divisions that occurred in the T cell subset by the time of harvest. (D) The experiment and calculation in (C) was carried out in a total of 6 experiments where the TCR stimulus was specific OVA antigen (n=3) or anti-CD3 antibody (n=3).

FIG. 3 demonstrates the role of IL-2 in CD25+ CD4+ T cell proliferation. (A) 3H-thymidine uptake by CD25+ or CD25+ CD4+ T cells alone (top), or T cells stimulated by CD86+ DCs not pulsed (middle) or pulsed (lower) with OVA protein ± IL-2 or PC61 anti-IL-2R mAb. (B) As in (A) but IL-2 effects on 3H-thymidine uptake and cell counts were assessed with time. (C) As in (A), but anti-IL-2R mAb or control rat IgG was added to CD25+ CD4+ T cells stimulated with DCs from wild type (WT) or IL-2−/− mice plus OVA peptide at 1 μg/ml for 3d. The numbers above the bars indicate the amount of IL-2 detected by ELISA in the same culture. (D) IL-2 production (ELISA) after stimulation with DC-OVA or DCs. Statistical significance was determined using the unpaired Student's t-test. *P<0.01.

FIG. 4 demonstrates Membrane costimulation of CD25+ CD4+ T cells by DCs. (A) Comparison of T cell responses to live (top, T:DC ratio of 1:1) or formaldehyde fixed (bottom, T:DC=1:3) CD86+ mature marrow DCs plus DO11.10 peptide at 1 μg/ml for 3d. Indicated concentration of anti-IL-2R Ab or control Ab were added to culture. Statistical significance was determined using the unpaired Student's t-test. *P<0.01. (B) Same as (A), but the activity of aldehyde-fixed DCs were studied with DCs that were charged with OVA (DC-OVA) or not (DC), and then added to CD25+ CD4+ and CD25− CD4+ T cells in the presence or absence of IL-2, with only the former subset responding to IL-2 in the absence of OVA (top left). (C) Marrow DCs (10⁴) were generated from wild type (WT) or CD80/CD86 knockout mice and matured in 50 ng/ml LPS prior to culture with CD25+ or CD25− CD4+ T cells (10⁴; purified from OT-II mice spleen and lymph node cells) for 3 days with or without 0.5 μg/ml OVA peptide. The degree of proliferation was assessed by incorporation of 3H-thymidine for the last 12 h. One representative result of three independent experiments is shown.

FIG. 5 demonstrates that CD25+ CD4+ T cells must contact DCs to proliferate actively. CFSE-labeled CD25+ CD4+ T cells (top) or CD25− CD4+ T cells (bottom) and the indicated stimuli were added to the inner and outer wells of transwell chambers, and the dilution of CFSE label per cell was followed by FACS after 3 days of culture. Dead cells were gated out by TOPRO-3 staining. One representative result of three independent experiments is shown.

FIG. 6 demonstrates CD25+ CD4+ T cells expanded by mature BMDCs retain phenotype and function. (A) Surface markers of CD25+ CD4+ and CD25− CD4+ T cells after 7d expansion by mature, CD86+ DC-OVA (closed lines, isotype control). (B) As in A, but the expression of the KJ1.16 clonotypic receptor in CD25+ CD4+ T cells is shown before and after 7 days of culture with DC-OVA. (C) 10⁴ DO11.10 T cells were cultured 7d with an equal number of OVA-pulsed CD86+ marrow DCs. CD11c+ DCs were eliminated by MACS, and then the recovered T cells were used to respond to 5×104 splenic APCs, or to suppress fresh CD25− CD4+ T cells in the presence of 1 μg/ml of OVA peptide (upper) or anti-CD3 mAb (lower). (D) CD25+ CD4+ T cells purified from DO11.10 mice were expanded with OVA-pulsed mature DCs for 7 days as in (C), with or without exogenous 100 U/ml IL-2. Fresh or cultured CD25+ CD4+ T cells were then mixed with freshly isolated CD25− CD4+ T cells from DO11.10 mice at the indicated ratios and cultured for 3 days. The degree of proliferation was assessed by incorporation of ³H thymidine for the last 12 h. Representative results of 3 or more similar experiments. Statistical significance was determined using the unpaired Student's t-test. *P<0.01.

FIG. 7 demonstrates CD25+ CD4+ T cells primarily proliferate to DCs as APCs. Proliferation was assessed by incorporation of 3H thymidine for last 12 h. (A) 10⁴ T cells were cultured 3 d with bone marrow DCs, spleen CD8+ or CD8− CD11c+ DCs matured by culture overnight in LPS, and CD19+ B cells matured in LPS. The APCs were exposed to 1 mg/ml OVA prior to use. Data with APCs lacking OVA were <10³ cpm and are omitted. (B) As in A, but bone marrow DCs were compared to spleen CD8+ or CD8− CD11c+ DCs, either fresh immature cells or matured by culture overnight, along with 1 μg/ml of DO11.10 peptide. (C) As in A, but DCs were compared to macrophages, either peritoneal exudate cells (PEC), thioglycollate elicited (TGC) PEC, or IFN-γ treated TGC-PEC. (D) CD25+ CD4+ T cells from DO11.10 mice were cultured for 3 d with lymph node CD11c+ DCs from untreated mice, or mice 5 days after CFA injection s.c. Representative results from 3 similar experiments.

FIG. 8 demonstrates DCs stimulate CD25+ CD4+ and CD25− CD4+ T cell proliferation in vivo. (A) CFSE-labeled T cells (0.7×10⁶) were injected i.v. and stimulated with marrow DCs or DC-OVA (2×10⁵) injected s.c. into the footpads 1 d later. Clonotype positive (KJ1.26+) TCR transgenic T cells (top, circle) were analyzed for proliferation and expression of CD25 three days later by dilution of the CFSE label in draining or distal (mesenteric) lymph nodes. (B) As in (A), but OVA antigen was delivered by the injection of 25 μg of soluble OVA into each footpad in the steady state. One representative result of 3 similar experiments.

FIG. 9 demonstrates NOD dendritic cell induction of growth of CD25+ CD4+ T cells from NOD.BDC2.5 or NOD mice. A) In vitro-derived NOD DCs were stained with antibodies specific for CD86 and MHC class II before (left) and after (right) magnetic bead enrichment of CD86+ cells. B) CD25+ CD4+ or CD25− CD4+ T cells sorted from BDC2.5 TCR transgenic mice were cultured with CD86+ NOD DCs with and without BDC peptide (30 ng ml-1) and IL-2. A 12-hour ³H-thymidine pulse was given on day 3. C) Same as B, but the dose of BDC peptide was 100 ng ml⁻¹ and the fold-increase in T cell numbers was monitored by counting on days 3, 5 and 7. One representative result of at least 3 experiments is shown.

FIG. 10 demonstrates the expansion of NOD CD25+CD4+ T cells with DCs and anti-CD3. A) CD25+ CD4+ T cells were isolated from NOD mice and cultured with NOD CD86+ DCs, with and without anti-CD3 and IL-2 as indicated. Proliferation was determined by ³H-thymidine incorporation on day 3. B) As in D, but cells were counted on days 3, 5, and 7, and the fold-increase in cell number calculated. One result of 2 similar experiments is shown. C) BDC2.5 TCR expression is high after stimulation with DC/BDC peptide but not DC/αCD3. BDC2.5 clonotype expression on CD25+ CD4+ T cells from BDC mice (left) or NOD (right) mice freshly isolated from spleen (top) or after expansion with DCs, IL-2 and BDC peptide (middle) or anti-CD3 (bottom). Mean fluorescence of clonotype staining is shown on each plot, and the isotype control peak is in grey.

FIG. 11 demonstrates BDC2.5 CD25+ CD4+ T cells proliferation in vivo. CFSE-labeled BDC2.5 CD25− CD4+ (left) or CD25+ CD4+ (right) T cells were injected into NOD mice. One day later, either DCs without antigen (top) or BDC peptide-pulsed DCs were injected s.c. Three days after antigen delivery, the injected >1000 CFSE-labeled clonotype positive cells from draining lymph nodes were assessed for proliferation by flow cytometry, gating on CD4+ lymphocytes.

FIG. 12 demonstrates enhanced DC-expanded CD25+ CD4+ T cells suppression of proliferation as compared to unexpanded CD25+ CD4+ T cells. A) CD25+ CD4+ T cells from NOD.BDC2.5 mice were expanded for 7 days with irradiated NOD DCs and BDC peptide and IL-2 as indicated. 10⁴ freshly isolated, sorted CD25− CD4+ T cells from BDC2.5 mice were cultured with NOD spleen cells, BDC peptide (30 ng/ml), and either freshly sorted CD25+ CD4+ or the indicated DC-expanded CD25+ CD4+ populations, at the ratios indicated. After 72 hr, proliferation was assessed by 3H-thymidine incorporation during a 12 hr pulse. One representative result from at least 3 is shown. B) Same as A, but both CD25+ and CD25− CD4+ T cells were isolated from NOD mice, and anti-CD3 was used as TCR stimulus instead of BDC peptide in both expansion and suppression cultures. One representative result from at least 3 is shown.

FIG. 13 demonstrates expanded CD25+ CD4+ T cells function in vivo to suppress development of diabetes. A) 4-6 week old NOD.BDC2.5 mice were given cyclophosphamide i.p. 3 days later, either 5×10⁵ DC-expanded CD25+ CD4+ T cells or CD25− CD4+ cells were injected i.v. B) NOD.scid females were injected with 3×10⁶ spleen cells from a diabetic NOD female and either nothing, or the indicated numbers of DC-expanded CD25+ CD4+ T or 3×10⁵ CD25− CD4+ cells from BDC2.5 mice. C) NOD.scid females were injected with either 4×10⁵ CD25− CD4+ cells from BDC2.5 mice, or 8×10⁶ spleen cells from a diabetic NOD female and either nothing, or the indicated numbers of DC-expanded CD25+ CD4+ T cells from BDC2.5 mice. The difference between diabetic spleen alone to db spleen+500 CD25+ CD4+ cells was significant, P=0.002, and diabetic spleen to db spleen+5000 CD25+ CD4+ cells P=0.002. One representative result from 2 experiments is shown. D) NOD.scid females were injected with 8×10⁶ diabetic spleen cells alone or with 10⁵ freshly isolated or DC/aCD3-expanded CD25+ CD4+ T cells from NOD mice. The number of mice in each group is indicated in parentheses.

FIG. 14 demonstrates that BDC2.5 CD25+ CD4+ T cells can still regulate diabetes when given after diabetogenic cells. NOD.scid females were injected with 8×10⁶ diabetic spleen cells, and 11 days later injected with either PBS, 10⁵, or 10⁴ DC-expanded CD25+ CD4+ T cells from BDC2.5 mice. The difference between diabetic spleen alone to diabetic spleen +10⁵, or 10⁴ DC-expanded CD25+ CD4+ cells was significant P=0.002. The number of mice in each group is indicated in parentheses.

FIG. 15 demonstrates that BDC2.5 regulatory T cells prevent diabtes in NOD mice. 13 week old NOD females were given PBS or the indicated cell populations. Diabetes was monitored weekly by urine glucose.

FIG. 16 demonstrates that BDC-peptide-expanded NOD regulatory T cells delay diabetes in NOD.scid mice. NOD.scid females were given 10⁷ spleen cells from diabetic mice plus either nothing, or NOD CD4+CD25+ cells stimulated with DCs and either anti-CD3 or BDC peptide as indicated. Diabetes was monitored by measuring urine glucose every 2-3 days.

FIG. 17 demonstrates uptake of islet cells by DCs. DCs were purified from bone marrow cultures, dissociated islet cells were purified from NOD mice, and the two populations were separately labeled with red (DCs) or green (islets) fluorescent dyes then mixed overnight at the indicated ratios and temperatures.

FIG. 18 demonstrates BDC2.5 regulatory cell response to islet-loaded DCs DCs were incubated overnight with islet cells at a ratio of 1:1 (high islet) or 3:1 (low islet), then washed and cultured with CD4+CD25+CD62L+ T cells from BDC2.5 mice. As controls, the same T cell population was also incubated with either DCs alone or DCs with BDCpeptide.

FIG. 19 demonstrates reversion of overt diabetes in NOD mice by treatment with GLP-1 and islet-specific Tregs. Blood glucose levels in diabetic mice given GLP-1 and insulin alone, indicated by open symbols, or GLP-1, insulin and 1.5×10⁶ DC-exp CD25+ CD62L+ cells from BDC2.5 mice, indicated by filled symbols. 3 of 5 of the latter group of mice were diabetes free for 100 days after treatment.

FIG. 20 demonstrates Treg-treated diabetic mouse response to glucose challenge. Blood glucose levels in glucose-challenged mice were measured at indicated times in 12-wk-non-diabetic NOD females (filled symbols), Treg-treated mice (open-symbols), or recently diabetic NOD females (crossed symbols). Treg treated mice returned to near normal glucose levels, (non-diabetic mice).

FIG. 21 demonstrates insulitis in Treg-treated diabetic mice. The number of islets in each group is indicated in parenthesis. Each islet was scored as having no insulitis (white), peri-insulitis (light grey), intra-insulitis with <60% infiltrate (dark grey), or intra-insulitis with >60% infiltrate (black). Only 25% of the islets from Treg treated mice had intra-insulitis.

### DETAILED DESCRIPTION OF THE PRESENT INVENTION

This invention provides, in one embodiment, an isolated culture-expanded T suppressor cell population, which expresses CD25 and CD4 on its cell surface, methods of producing the same, and methods of use thereof.

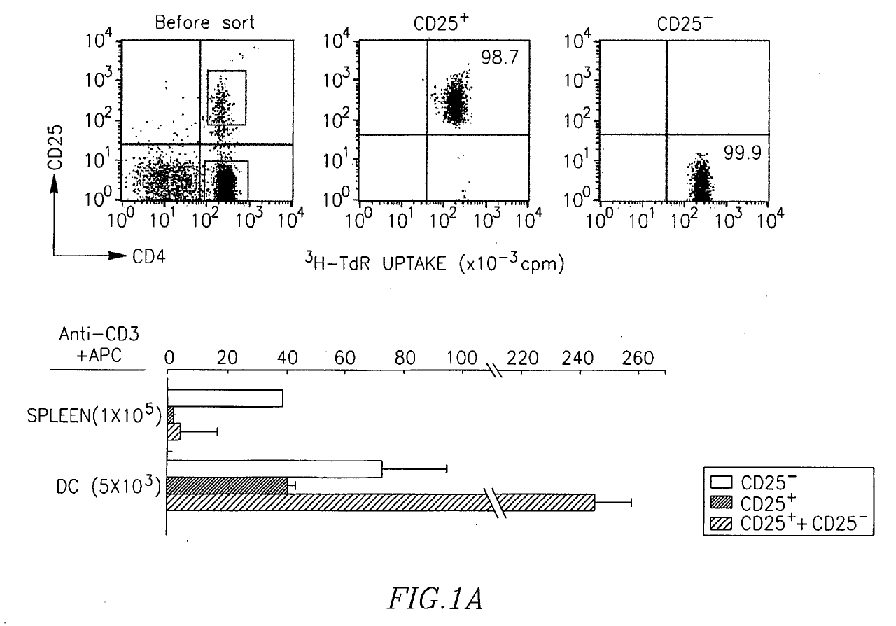

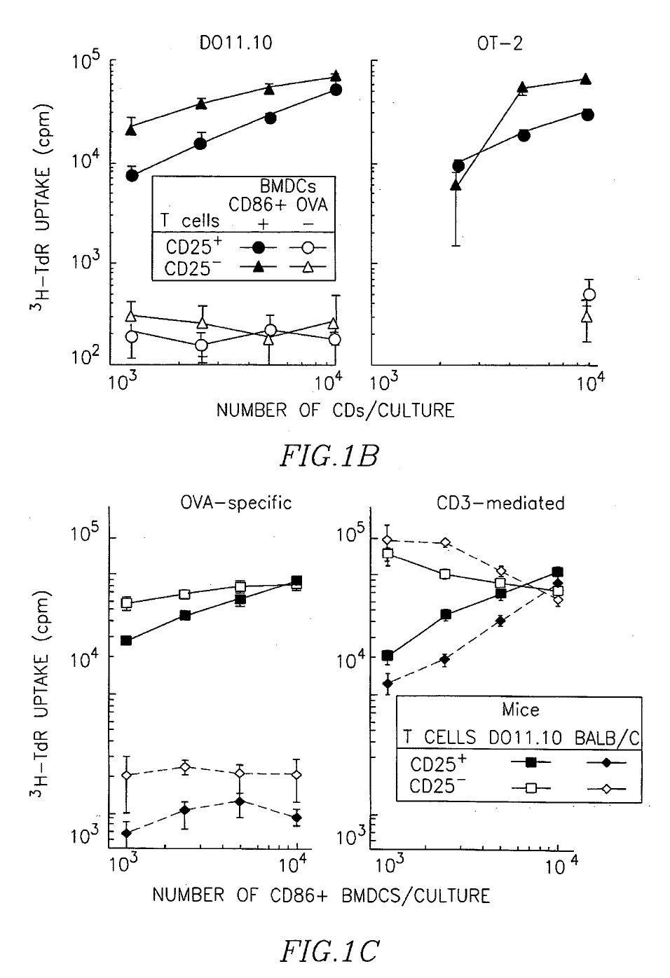

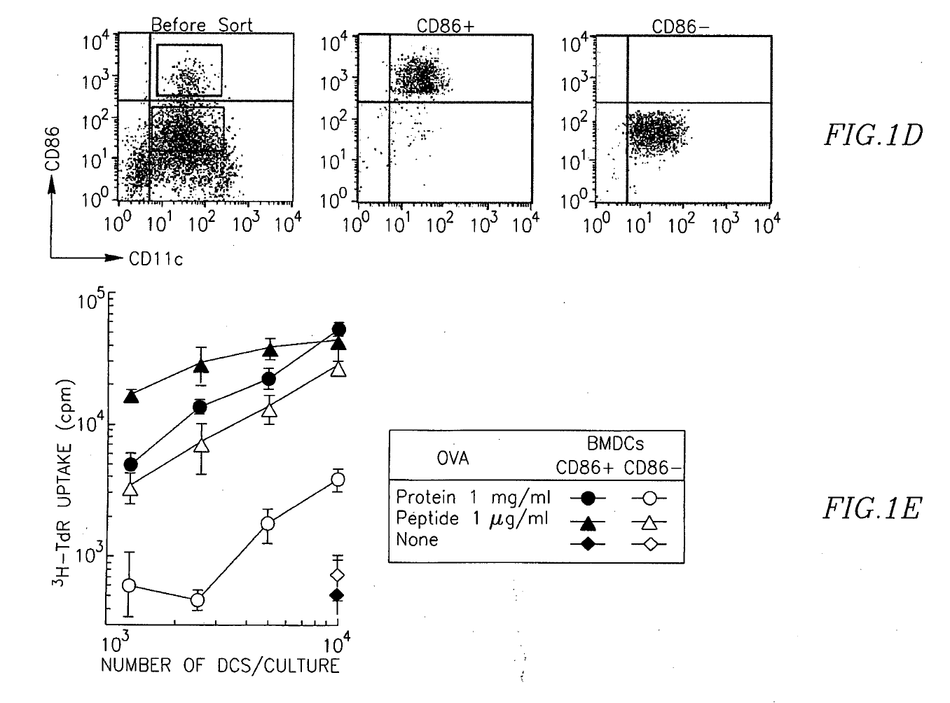

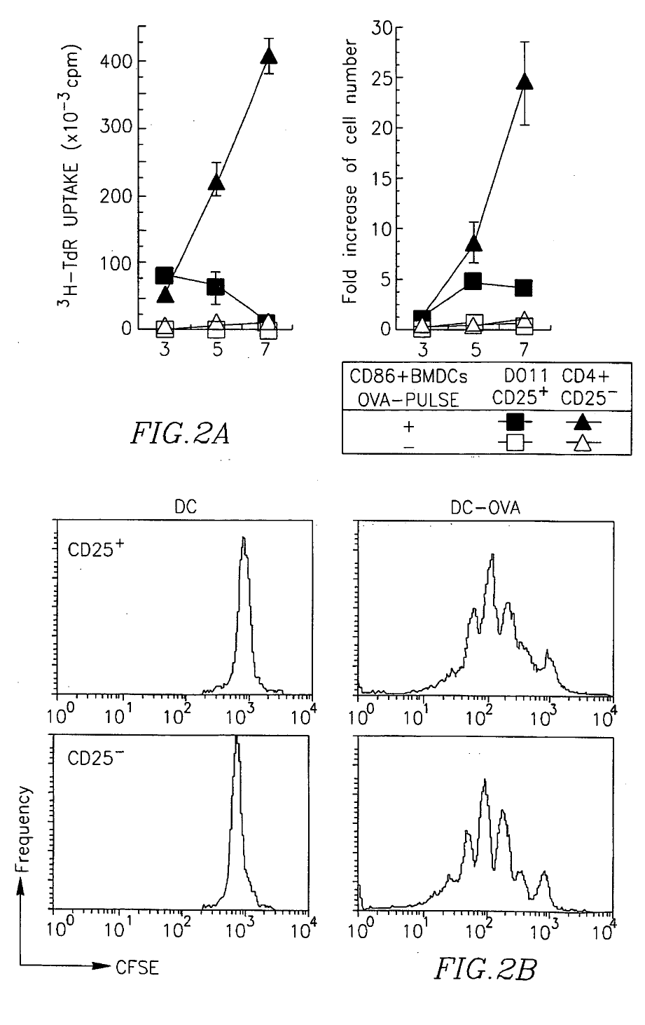

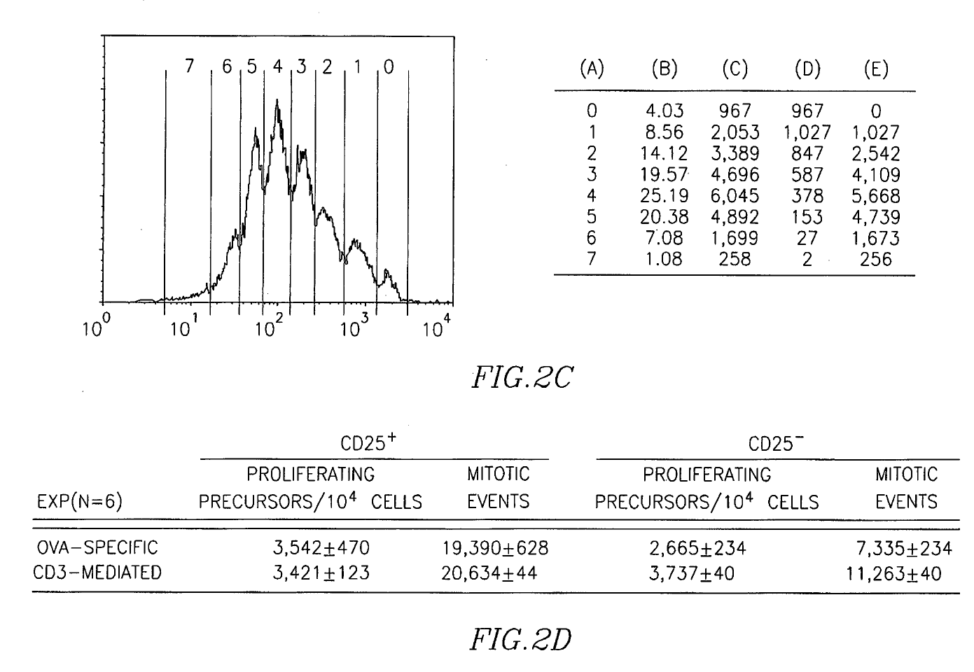

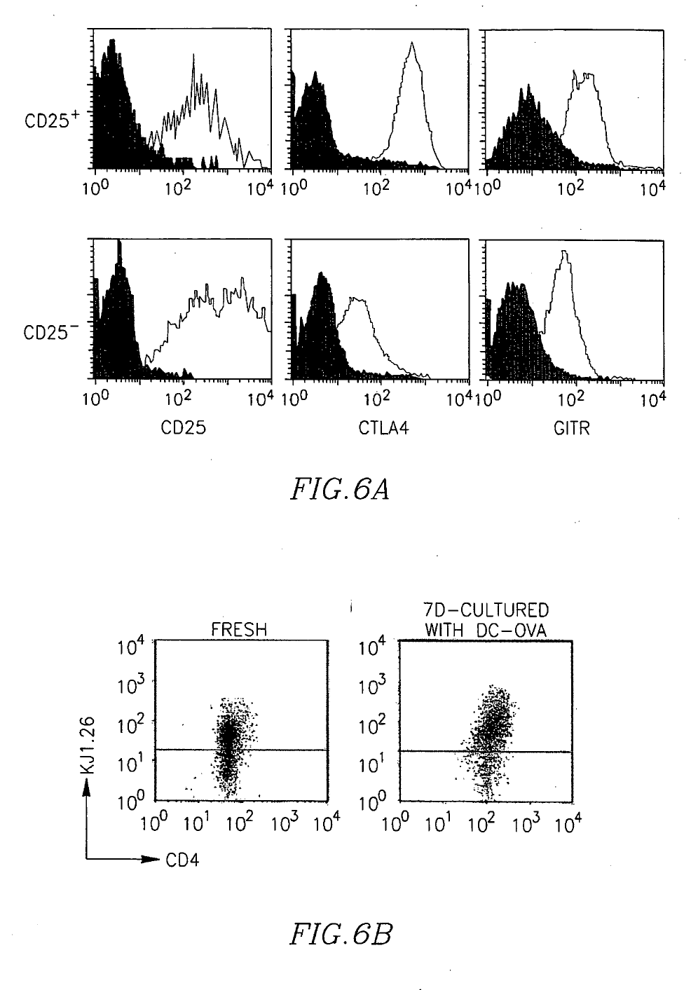

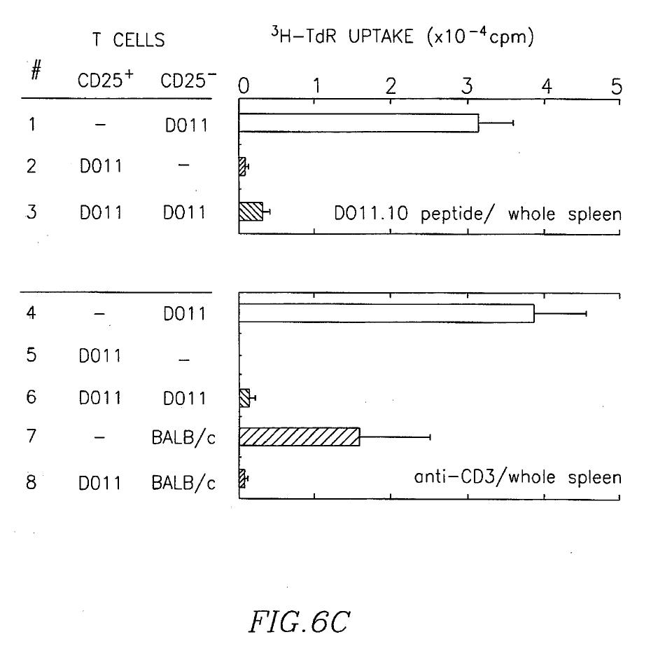

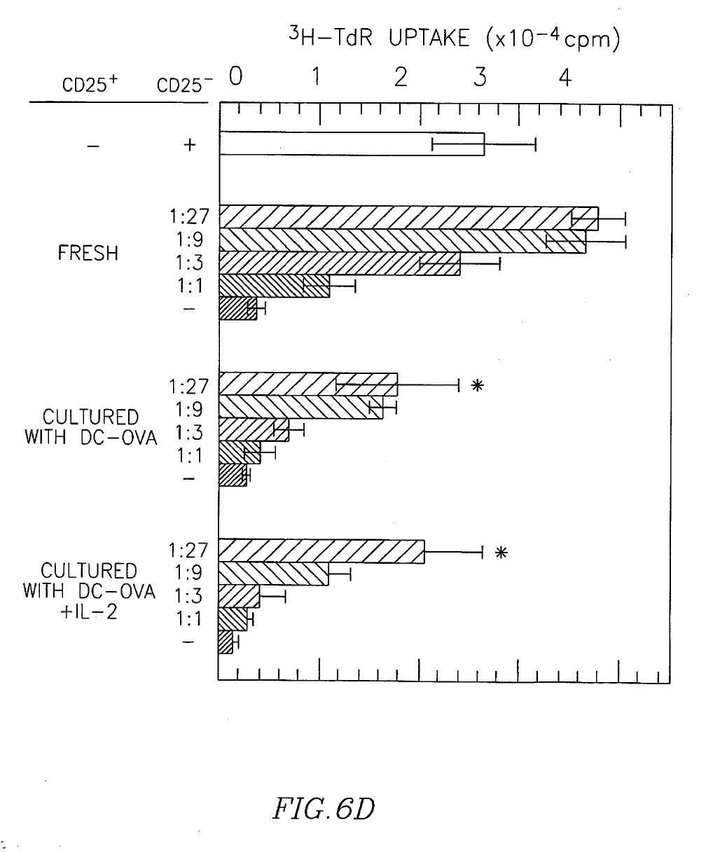

Isolated, culture-expanded T suppressor cells expressing CD25 and CD4 were obtained herein following their incubation with dendritic cells (FIG. 1), with T suppressor cells driven into multiple cell cycles (FIG. 2). T suppressor cell expansion did not alter their phenotype, nor abrogate their function (FIG. 6).

In one embodiment, this invention provides an isolated, culture-expanded T suppressor cell population, wherein the population expresses CD25 and CD4 on its cell surface.

In one embodiment, the phrase “T suppressor cell” or “suppressor T cell”, or “regulatory T cell”, refers to a T cell population that inhibits or prevents the activation, or in another embodiment, the effector function, of another T lymphocyte. In one embodiment, the T suppressors are a homogenous population, or in another embodiment, a heterogeneous population.

The T suppressor cells of this invention express CD25 and CD4 on their cell surface. In one embodiment, the T suppressor cells may be classified as CD25^{high} expressors, or in another embodiment, the T suppressor cells may be classified as CD4^{low} expressors, or in another embodiment, a combination thereof In another embodiment, the T suppressor cells may express CTLA-4, or in another embodiment, GITR. In one embodiment, the T suppressor cells may be classified as CTLA-4^{high} expressors, or in another embodiment, the T suppressor cells may be classified as GITR^{high}, or in another embodiment, a combination thereof In another embodiment, the T suppressor cells of this invention are CD69⁻. In another embodiment, the T suppressor cells of this invention are CD62L^{hi}, CD45RB^{lo}, CD45RO^{hi}, CD45RA⁻, α_{E}β₇ integrin, Foxp3, expressors, or any combination thereof It is to be understood that the isolated culture-expanded T suppressor cells of this invention may express any number or combination of cell surface markers, as described herein, and as is well known in the art, and are to be considered as part of this invention.

In one embodiment, the T suppressor cells of this invention express the CD62L antigen, which in one embodiment, is a 74 kDa glycoprotein, and in another embodiment, is a member of the selectin family of cell surface molecules. In another embodiment, the phrase “CD62L” may also be referred to as “L-selectin”, “LECAM-1”, or “LAM-1”, all of which are to be considered synonymous herein. CD62L binds a series of glycoproteins, in other embodiments, including CD34, GlyCAM-1 and MAdCAM-1. CD62L is important, in another embodiment, for homing of the lymphocytes via the high endothelial venules to peripheral lymph nodes and Peyer's patches, where in another embodiment, they may carry out their effector function, for example, and in one embodiment, suppression of autoimmune responses. The CD62L antigen also contributes, in another embodiment, to the recruitment of leukocytes from the blood to areas of inflammation, and in another embodiment, recruited cells may thereby be induced to become suppressor cells.

In one embodiment, the T suppressor cells of this invention are obtained by positive selection for expression of CD4 and CD25, and in another embodiment, the T suppressor cells may also be selected for the absence of CD45RA expression, i.e. negative selection procedures, as are well known in the art. In another embodiment, other markers can be used to further separate subpopulations of the T suppressor cells, including CD69, CCR6, CD30, CTLA-4, CD62L, CD45RB, CD45RO, Foxp3, or a combination thereof.

In one embodiment, the T suppressor cells of this invention may be obtained from in vivo sources, such as, for example, peripheral blood, leukopheresis blood product, apheresis blood product, peripheral lymph nodes, gut associated lymphoid tissue, spleen, thymus, cord blood, mesenteric lymph nodes, liver, sites of immunologic lesions, e.g. synovial fluid, pancreas, cerebrospinal fluid, tumor samples, granulomatous tissue, or any other source where such cells may be obtained In one embodiment, the T suppressor cells are obtained from human sources, which may be, in another embodiment, from human fetal, neonatal, child, or adult sources. In another embodiment, the T suppressor cells of this invention may be obtained from animal sources, such as, for example, porcine or simian, or any other animal of interest. In another embodiment, the T suppressor cells of this invention may be obtained from subjects that are normal, or in another embodiment, diseased, or in another embodiment, susceptible to a disease of interest.

In one embodiment, the T suppressor cells and/or dendritic cells, as described further hereinbelow, of this invention are isolated from tissue, and, in another embodiment, an appropriate solution may be used for dispersion or suspension, toward this end. In another embodiment, T suppressor cells and/or dendritic cells, as described further hereinbelow, of this invention may be cultured in solution.

Such a solution may be, in another embodiment, a balanced salt solution, such as normal saline, PBS, or Hank's balanced salt solution, or others, each of which represents another embodiment of this invention. The solution may be supplemented, in other embodiments, with fetal calf serum, bovine serum albumin (BSA), normal goat serum, or other naturally occurring factors, and, in another embodiment, may be supplied in conjunction with an acceptable buffer. The buffer may be, in other embodiments, HEPES, phosphate buffers, lactate buffers, or the like, as will be known to one skilled in the art.

In another embodiment, the solution in which the T suppressor cells or dendritic cells of this invention may be placed is in medium is which is serum-free, which may be, in another embodiment, commercially available, such as, for example, animal protein-free base media such as X-VIVO 10™ or X-VIVO 15™ (BioWhittaker, Walkersville, Md.), Hematopoietic Stem Cell-SFM media (GibcoBRL, Grand Island, N.Y.) or any formulation which promotes or sustains cell viability. Serum-free media used, may, in another emodiment, be as those described in the following patent documents: WO 95/00632; U.S. Pat. No. 5,405,772; PCT US94/09622. The serum-free base medium may, in another embodiment, contain clinical grade bovine serum albumin, which may be, in another embodiment, at a concentration of about 0.5-5%, or, in another embodiment, about 1.0% (w/v). Clinical grade albumin derived from human serun, such as Buminate® (Baxter Hyland, Glendale, Calif.), may be used, in another embodiment.

In another embodiment, the T suppressor cells of this invention may be separated via affinity-based separation methods. Techniques for affinity separation may include, in other embodiments, magnetic separation, using antibody-coated magnetic beads, affinity chromatography, cytotoxic agents joined to a monoclonal antibody or use in conjunction with a monoclonal antibody, for example, complement and cytotoxins, and “panning” with an antibody attached to a solid matrix, such as a plate, or any other convenient technique. In other embodiment, separation techniques may also include the use of fluorescence activated cell sorters, which can have varying degrees of sophistication, such as multiple color channels, low angle and obtuse light scattering detecting channels, impedance channels, etc. It is to be understood that any technique, which enables separation of the T suppressor cells of this invention may be employed, and is to be considered as part of this invention.

In another embodiment, the affinity reagents employed in the separation methods may be specific receptors or ligands for the cell surface molecules indicated hereinabove. In other embodiments, peptide-MHC antigen and T cell receptor pairs may be used; peptide ligands and receptor, effector and receptor molecules, or others. Antibodies and T cell receptors may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art.

In another embodiment, the antibodies utilized herein may be conjugated to a label, which may, in another embodiment, be used for separation. Labels may include, in other embodiments, magnetic beads, which allow for direct separation, biotin, which may be removed with avidin or streptavidin bound to, for example, a support, fluorochromes, which may be used with a fluorescence activated cell sorter, or the like, to allow for ease of separation, and others, as is well known in the art. Fluorochromes may include, in one embodiment, phycobiliproteins, such as, for example, phycoerythrin, allophycocyanins, fluorescein, Texas red, or combinations thereof. In one embodiment, antibodies are labeled In one embodiment suppressors can be purified by positive or negative selection.

In one embodiment, cell separations utilizing antibodies will entail the addition of an antibody to a suspension of cells, for a period of time sufficient to bind the available cell surface antigens. The incubation may be for a varied period of time, such as in one embodiment, for 5 minutes, or in another embodiment, 15 minutes, or in another embodiment, 30 minutes. Any length of time which results in specific labeling with the antibody, with minimal non-specific binding is to be considered envisioned for this aspect of the invention.

In another embodiment, the staining intensity of the cells can be monitored by flow cytometry, where lasers detect the quantitative levels of fluorochrome (which is proportional to the amount of cell surface antigen bound by the antibodies). Flow cytometry, or FACS, can also be used, in another embodiment, to separate cell populations based on the intensity of antibody staining, as well as other parameters such as cell size and light scatter.

In another embodiment, the labeled cells are separated based on their expression of CD4 and CD25. In another embodiment, the cells may be further separated based on their expression of CD62L. The separated cells may be collected in any appropriate medium that maintains cell viability, and may, in another embodiment, comprise a cushion of serum at the bottom of the collection tube.

In another embodiment, the culture containing the T suppressor cells of this invention may contain cytokines or growth factors to which the cells are responsive. -In one embodiment, the cytokines or growth factors promote survival, growth, function, or a combination thereof of the T suppressor cells. Cytokines and growth factors may include, in other embodiment, polypeptides and non-polypeptide factors. In one embodiment, the cytokines may comprise interleukins.

In one embodiment, the isolated culture-expanded T suppressor cell populations of this invention are antigen specific.

In one embodiment, the term “antigen specific” refers to a property of the population such that supply of a particular antigen, or in another embodiment, a fragment of the antigen, results, in one embodiment, in specific suppressor cell proliferation, when presented the antigen, in the context of MHC. In another embodiment, supply of the antigen or fragment thereof, results in suppressor cell production of interleukin 2, or in another embodiment, enhanced expression of the T cell receptor (TCR) on its surface, or in another embodiment, suppressor cell function. In one embodiment, the T suppressor cell population expresses a monoclonal T cell receptor. In another embodiment, the T suppressor cell population expresses polyclonal T cell receptors.

In one embodiment, the T suppressor cells will be of one or more specificities, and may include, in another embodiment, those that recognize a mixture of antigens derived from an antigenic source, such as, for example, in diabetes, where recognition of a pancreatic beta cell line or islet tissue itself may be used to expand the T suppressor cells. In one embodiment suppressors can be purified by positive or negative selection.

In another embodiment, the antigen is a self-antigen. In one embodiment, the term “self-antigen” refers to an antigen that is normally expressed in the body from which the suppressor T cell population is derived. In another embodiment, self-antigen is comparable to one, or, in another embodiment, indistinct from one normally expressed in a body from which the suppressor T cell population is derived, though may not directly correspond to the antigen. In another embodiment, self-antigen refers to an antigen, which when expressed in a body, may result in the education of self-reactive T cells. In one embodiment, self-antigen is expressed in an organ that is the target of an autoimmune disease. In one embodiment, the self-antigen is expressed in a pancreas, thyroid, connective tissue, kidney, lung, digestive system or nervous system. In another embodiment, self-antigen is expressed on pancreatic β cells.

In another embodiment, a library of peptides that span an antigenic protein is used in this invention. In one embodiment, the peptides are about 15 amino acids in length, and may, in another embodiment, be staggered every 4 amino acids along the length of the antigenic protein.

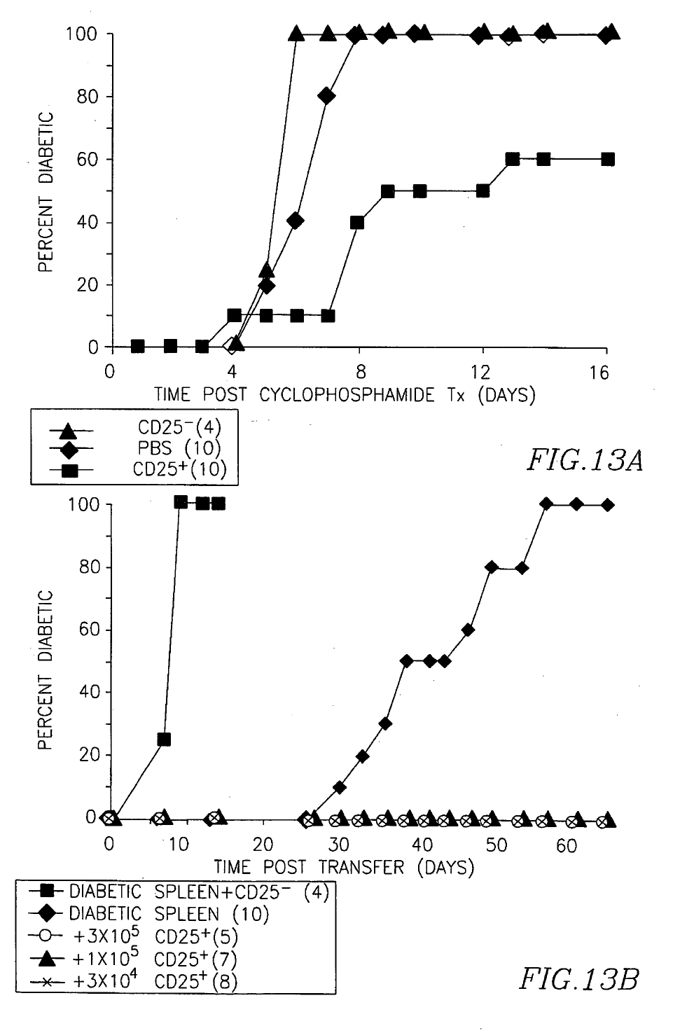

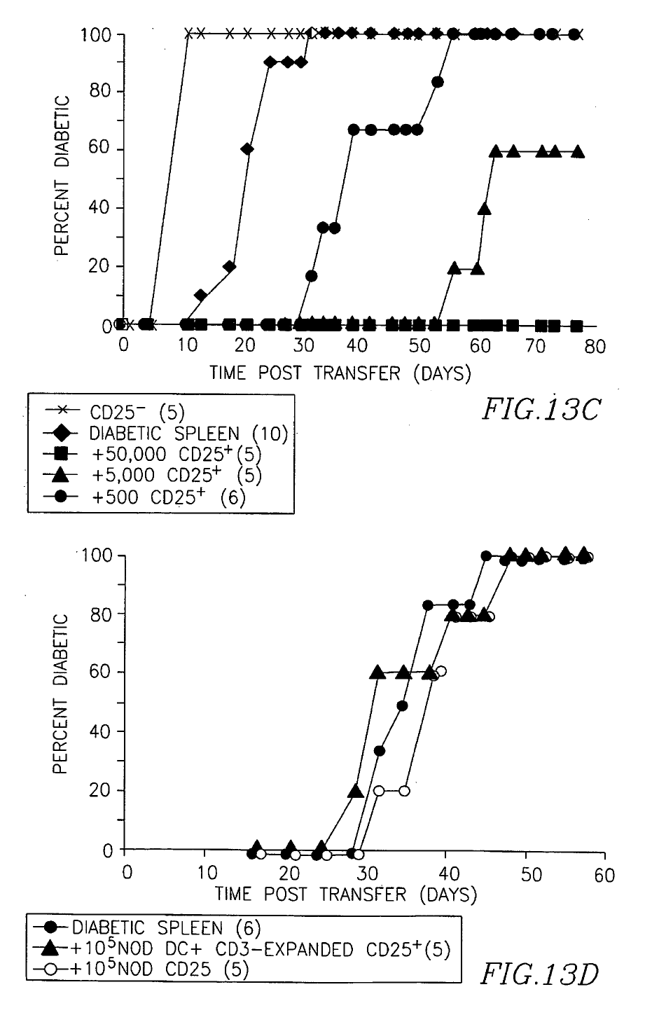

In one embodiment, the isolated culture-expanded T suppressor cell population suppresses an autoimmune response. In one embodiment, the term “autoimmune response” refers to an immune response directed against an auto- or self-antigen. In one embodiment, the autoimmune response is rheumatoid arthritis, multiple sclerosis, diabetes mellitus, myasthenia gravis, pernicious anemia, Addison's disease, lupus erythematosus, Reiter's syndrome, atopic dermatitis, psoriasis or Graves disease. In one embodiment, the autoimmune disease caused in the subject is a result of self-reactive T cells, which recognize multiple self-antigens. In one embodiment, the T suppressor cell populations of this invention may be specific for a single self-antigen in a disease where multiple self-antigens are recognized, yet the T suppressor cell population effectively suppresses the autoimmune disease. Such a phenomenon was exemplified herein, for example, in FIG. 13, where DC expanded CD25+ CD4+ suppressor T cells into NOD mice rendered diabetic with diabetic spleen cells, prevented the development of diabetes, which is a disease wherein auto-reactive T cells recognize multiple self-antigens.

In another embodiment, the antigen may be any molecule recognized by the immune system of the mammal as foreign. For example, the antigen may be any foreign molecule, such as a protein (including a modified protein such as a glycoprotein, a mucoprotein, etc.), a nucleic acid, a carbohydrate, a proteoglycan, a lipid, a mucin molecule, or other similar molecule, including any combination thereof. The antigen may, in another embodiment, be a cell or a part thereof, for example, a cell surface molecule. In another embodiment, the antigen may derive from an infectious virus, bacteria, fungi, or other organism (e.g., protists), or part thereof. These infectious organisms may be active, in one embodiment or inactive, in another embodiment, which may be accomplished, for example, through exposure to heat or removal of at least one protein or gene required for replication of the organism.

In one embodiment, the term “antigen” refers to a protein, or peptide, associated with a particular disease for which the cells of this invention are being used to modulate, or for use in any of the methods of this invention. In one embodiment, the term “antigen” may refer to a synthetically derived molecule, or a naturally derived molecule, which shares sequence homology with an antigen of interest, or structural homology with an antigen of interest, or a combination thereof In one embodiment, the antigen may be a mimetope.

In another embodiment, isolated culture-expanded T suppressor cell population suppresses an inflammatory response. In one embodiment, the term “inflammatory disorder” refers to any disorder that is, in one embodiment, caused by an “inflammatory response” also referred to, in another embodiment, as “inflammation” or, in another embodiment, whose symptoms include inflammation. By way of example, an inflammatory disorder caused by inflammation may be a septic shock, and an inflammatory disorder whose symptoms include inflammation may be rheumatoid arthritis. The inflammatory disorders of the present invention comprise, in another embodiment, cardiovascular disease, rheumatoid arthritis, multiple sclerosis, Crohn's disease, inflammatory bowel disease, systemic lupus erythematosis, polymyositis, septic shock, graft versus host disease, host versus graft disease, asthma, rhinitis, psoriasis, cachexia associated with cancer, or eczema In one embodiment, as described hereinabove, the inflammation in the subject may be a result of T cells, which recognize multiple antigens in the subject. In one embodiment, the T suppressor cell populations of this invention may be specific for a single antigen where multiple antigens are recognized, yet the T suppressor cell population effectively suppresses the inflammation in the subject.

In another embodiment, the isolated culture-expanded T suppressor cell populations of this invention suppress an allergic response. In one embodiment, the term “allergic response” refers to an immune system attack against a generally harmless, innocuous antigen or allergen. Allergies may in one embodiment include, but are not limited to, hay fever, asthma, atopic eczema as well as allergies to poison oak and ivy, house dust mites, bee pollen, nuts, shellfish, penicillin or other medications, or any other compound or compounds which induce an allergic response. In one embodiment, multiple allergens elicit an allergic response, and the antigen recognized by the T suppressor cells of this invention may be any antigen thereof.

In another embodiment, the isolated culture-expanded T suppressor cell population downmodulates an immune response. In one embodiment, an immune response to a particular antigen may be beneficial to the host, such as, for example, a response directed against an antigen from a pathogen that has invaded the subject. In one embodiment, such an immune response may be too robust, such that even after the pathogen has been eradicated, or controlled, the immune response is sustained and causes damage to the host, such as, for example, by causing tissue necrosis, in tissue which formerly was infected with the pathogen. In these and other circumstances, the isolated culture-expanded T suppressor cell population may be useful in downmodulating an immune response, such that the host is not compromised in any way by the persistence of such an immune response.

In another embodiment, the immune response, whose downmodulation is desired is host versus graft disease. With the improvement in the efficiency of surgical techniques for transplanting tissues and organs such as skin, kidney, liver, heart, lung, pancreas and bone marrow to subjects, perhaps the principal outstanding problem is the immune response mounted by the recipient to the transplanted allograft or organ, often resulting in rejection. When allogeneic cells or organs are transplanted into a host (i.e, the donor and receipient are different individual from the same species), the host immune system is likely to mount an immune response to foreign antigens in the transplant (host-versus-graft disease) leading to destruction of the transplanted tissue. Accordingly, the isolated culture-expanded T suppressor cell population may be used, in one embodiment, to prevent such rejection of transplanted tissue or organ.

In another embodiment, the immune response, whose downmodulation is desired is graft versus host disease (GVHD). GVHD is a potentially fatal disease that occurs when immunologically competent cells are transferred to an allogeneic recipient. In this situation, the donor's immunocompetent cells may attack tissues in the recipient. Tissues of the skin, gut epithelia and liver are frequent targets and may be destroyed during the course of GVHD. The disease presents an especially severe problem when immune tissue is being transplanted, such as in bone marrow transplantation; but less severe GVHD has also been reported in other cases as well, including heart and liver transplants. The isolated culture-expanded T suppressor cell population may be used, in one embodiment, to preventing or ameliorating such disease.

It is to be understood that the downmodulation of any immune response, via the use of the isolated culture-expanded T suppressor cell populations of this invention are to be considered as part of this invention, and an embodiment thereof.

In one embodiment, the isolated culture-expanded T suppressor cell populations secrete substances, which mediate the suppressive effects. In one embodiment, the T suppressor cells of this invention mediate bystander suppression, without a need for direct cell contact. In one embodiment, the substances mediating suppression secreted by the T suppressor cell populations of this invention may include IL-10, TGF-β, or a combination thereof.

In another embodiment, the isolated culture-expanded T suppressor cell populations may be engineered to express substances which when secreted mediate suppressive effects, such as, for example, the cytokines listed hereinabove. In another embodiment, the isolated culture-expanded T suppressor cell populations may be engineered to express particular adhesion molecules, or other targeting molecules, which, when the cells are provided to a subject, facilitate targeting of the T suppressor cell populations to a site of interest. For example, when T suppressor cell activity is desired to downmodulate or prevent an immune response at a mucosal surface, the isolated culture-expanded T suppressor cell populations of this invention may be further engineered to express the αₑβ₇ adhesion molecule, which has been shown to play a role in mucosal homing. The cells can be engineered to express other targeting molecules, such as, for example, an antibody specific for a protein expressed at a particular site in a tissue, or, in another embodiment, expressed on a particular cell located at a site of interest, etc. Numerous methods are well known in the art for engineering the cells, and may comprise the use of a vector, or naked DNA, wherein a nucleic acid coding for the targeting molecule of interest is introduced via any number of methods well described.

A nucleic acid sequence of interest may be subcloned within a particular vector, depending upon the desired method of introduction of the sequence within cells. Once the nucleic acid segment is subcloned into a particular vector it thereby becomes a recombinant vector. Polynucleotide segments encoding sequences of interest can be ligated into commercially available expression vector systems suitable for transducing/transforming mammalian cells and for directing the expression of recombinant products within the transduced cells. It will be appreciated that such commercially available vector systems can easily be modified via commonly used recombinant techniques in order to replace, duplicate or mutate existing promoter or enhancer sequences and/or introduce any additional polynucleotide sequences such as for example, sequences encoding additional selection markers or sequences encoding reporter polypeptides.

There are a number of techniques known in the art for introducing the above described recombinant vectors into cells, such as, but not limited to: direct DNA uptake techniques, and virus, plasmid, linear DNA or liposome mediated transduction, receptor-mediated uptake and magnetoporation methods employing calcium-phosphate mediated and DEAE-dextan mediated methods of introduction, electroporation, liposome-mediated transfection, direct injection, and receptor-mediated uptake (for further detail see, for example, “Methods in Enzymology” Vol. 1-317, Academic Press, Current Protocols in Molecular Biology, Ausubel F. M. et al. (eds.) Greene Publishing Associates, (1989) and in Molecular Cloning: A Laboratory Manual, 2nd Edition, Sambrook et al. Cold Spring Harbor Laboratory Press, (1989), or other standard laboratory manuals). Bombardment with nucleic acid coated particles is also envisaged.

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay. If the gene product of interest to be expressed by a cell is not readily assayable, an expression system can first be optimized using a reporter gene linked to the regulatory elements and vector to be used. The reporter gene encodes a gene product, which is easily detectable and, thus, can be used to evaluate efficacy of the system. Standard reporter genes used in the art include genes encoding β-galactosidase, chloramphenicol acetyl transferase, luciferase and human growth hormone, or any of the marker proteins listed herein.

In another embodiment, this invention provides a method for producing an isolated, culture-expanded T suppressor cell population, comprising contacting CD25+ CD4+ T cells with dendritic cells and an antigenic peptide, an antigenic protein or an agent that cross-links a T cell receptor on said T cells in a culture, for a period of time resulting in antigen-specific CD25+ CD4+ T cell expansion and isolating the expanded CD25+ CD4+ T cells thus obtained, thereby producing an isolated, culture-expanded T suppressor cell population.

In one embodiment, the method for producing an isolated culture-expanded T suppressor cell population, further comprises the step of adding a cytokine or growth factor to the dendritic cell, CD25+ CD4+ T cell culture. In one embodiment, the cytokine is interleukin-2, or any other cytokine or growth factor desired.

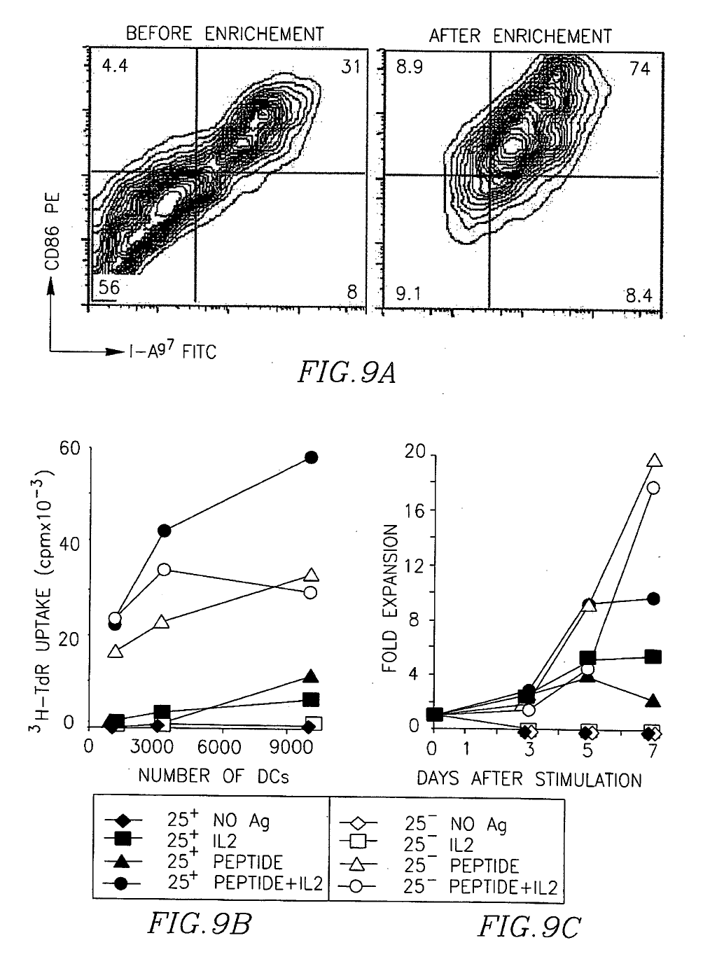

Dendritic cells stimulated CD25+ CD4+ T cell proliferation, as exemplified herein, in FIG. 1 and FIG. 9. While spleen cells used as antigen presenting cells resulted in CD25+ CD4+ T cell anergy, when stimulated with anti-CD3 antibody, the use of dendritic cells resulted in proliferation is response to anti-CD3 antibody, OVA antigen, in CD25+ CD4+ T cells from OVA-TCR transgenic mice, BDC peptide in CD25+ CD4+ T cells from BDC2.5 TCR transgenic mice, and CD25+ CD4+ T cells from NOD mice.

In one embodiment, the term “dendritic cell” (DC) refers to antigen-presenting cells, which are capable of presenting antigen to T cells, in the context of MHC. In one embodiment, the dendritic cells utilized in the methods of this invention may be of any of several DC subsets, which differentiate from, in one embodiment, lymphoid or, in another embodiment, myeloid bone marrow progenitors. In one embodiment, DC development may be stimulated via the use of granulocyte-macrophage colony-stimulating-factor (GM-CSF), or in another embodiment, interleukin (IL)-3, which may, in another embodiment, enhance DC survival.

In another embodiment, DCs may be generated from proliferating progenitors isolated from bone marrow, as exemplified herein. In another embodiment, DCs may be isolated from CD34+ progenitors as described by Caux and Banchereau in Nature in 1992, or from monocytes, as described by Romani et al, J. Exp. Med. 180: 83-93 '94 and Bender et al, J. Immunol. Methods, 196: 121-135, '96 1996. In another embodiment, the DCs are isolated from blood, as described for example, in O'Doherty et al, J. Exp. Med. 178: 1067-1078 1993 and Immunology 82: 487-493 1994, all methods of which are incorporated fully herewith by reference.

In one embodiment, the DCs utilized in the methods of this invention may express myeloid markers, such as, for example, CD11c or, in another embodiment, an IL-3 receptor-α (IL-3Rα) chain (CD123). In another embodiment, the DCs may produce type I interferons (IFNs). In one embodiment, the DCs utilized in the methods of this invention express costimulatory molecules. In another embodiment, the DCs utilized in the methods of this invention may express additional adhesion molecules, which may, in one embodiment, serve as additional costimulatory molecules, or in another embodiment, serve to target the DCs to particular sites in vivo, when delivered via the methods of this invention, as described further hereinbelow.

In one embodiment, the DCs may be obtained from in vivo sources, such as, for example, most solid tissues in the body, peripheral blood, lymph nodes, gut associated lymphoid tissue, spleen, thymus, skin, sites of immunologic lesions, e.g. synovial fluid, pancreas, cerebrospinal fluid, tumor samples, granulomatous tissue, or any other source where such cells may be obtained. In one embodiment, the dendritic cells are obtained from human sources, which may be, in another embodiment, from human fetal, neonatal, child, or adult sources. In another embodiment, the dendritic cells used in the methods of this invention may be obtained from animal sources, such as, for example, porcine or simian, or any other animal of interest. In another embodiment, dendritic cells used in the methods of this invention may be obtained from subjects that are normal, or in another embodiment, diseased, or in another embodiment, susceptible to a disease of interest.

Dendritic cell separation may accomplished in another embodiment, via any of the separation methods as described herein. In one embodiment, positive and/or negative affinity based selections are conducted. In one embodiment, positive selection is based on CD86 expression, and negative selection is based on GRI expression.

In another embodiment, the dendritic cells used in the methods of this invention may be generated in vitro by culturing monocytes in presence of GM-CSF and IL-4.

In one embodiment, the dendritic cells used in the methods of this invention may express CD83, an endocytic receptor to increase uptake of the autoantigen such as DEC-205/CD205 in one embodiment, or DC-LAMP (CD208) cell surface markers, or, in another embodiment, varying levels of the antigen presenting MHC class I and II products, or in another embodiment, accessory (adhesion and co-stimulatory) molecules including CD40, CD54, CD58 or CD86, or any combination thereof. In another embodiment, the dendritic cells may express varying levels of CD115, CD14, CD68 or CD32.

In one embodiment, mature dendritic cells are used for the methods of this invention. In one embodiment, the term “mature dendritic cells” refers to a population of dendritic cells with diminished CD115, CD14, CD68 or CD32 expression, or in another embodiment, a population of cells with enhanced CD86 expression, or a combination thereof. In another embodiment, mature dendritic cells will exhibit increased expression of one or more of p55, CD83, CD40 or CD86 or a combination thereof. In another embodiment, the dendritic cells used in the methods of this invention will express the DEC-205 receptor on their surface. In another embodiment, maturation of the DCs may be accomplished via, for example, CD40 ligation, CpG oligodeoxyribonucleotide addition, ligation of the IL-1, TNFα or TOLL like receptor ligand, bacterial lipoglycan or polysaccharide addition or activation of an intracellular pathway such as TRAF-6 or NF-κB.

In one embodiment, inducing DC maturation may be in combination with endocytic receptor delivery of a preselected antigen. In one embodiment, endocytic receptor delivery of antigen may be via the use of the DEC-205 receptor.

In one embodiment, the maturation status of the dendritic may be confirmed, for example, by detecting either one or more of 1) an increase expression of one or more of p55, CD83, CD40 or CD86 antigens; 2) loss of CD115, CD14, CD32 or CD68 antigen; or 3) reversion to a macrophage phenotype characterized by increased adhesion and loss of veils following the removal of cytokines which promote maturation of PBMCs to the immature dendritic cells, by methods well known in the art, such as, for example, immunohistochemistry, FACS analysis, and others.

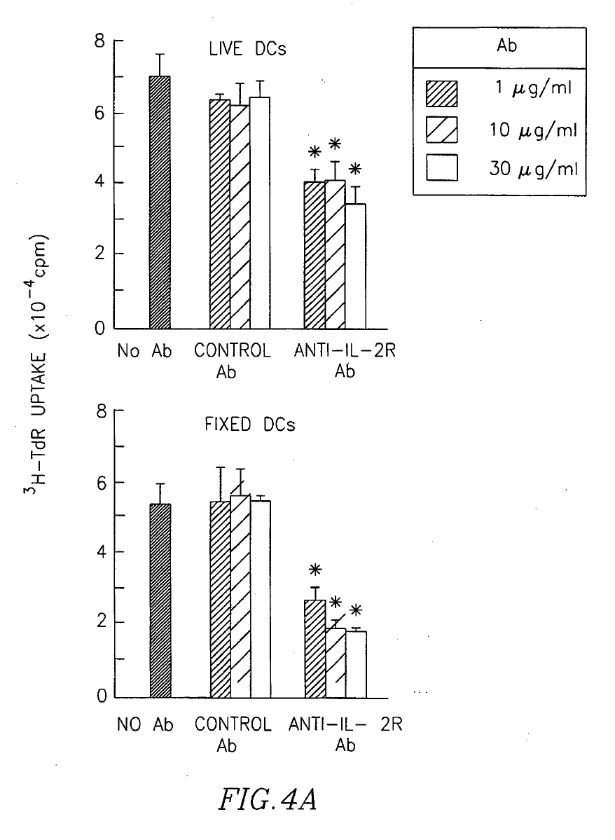

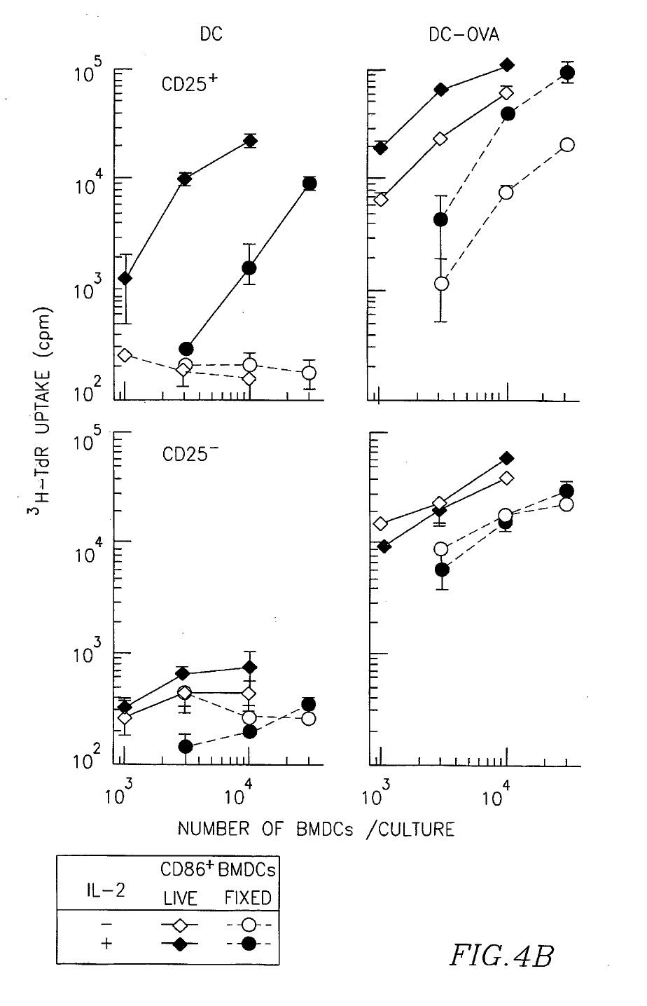

Dendritic cells prepared from mice genetically deleted for CD80 and CD86 (B7-1 and B7-2) were demonstrated to be less efficient at stimulating proliferation of CD25+ CD4+ T cells (FIG. 4), playing a role in of CD25+ CD4+ T suppressor cell expansion. In one embodiment, the dendritic cells used for the methods of this invention may express, or in another embodiment, may be engineered to express a costimulatory molecule. In one embodiment, dendritic cells used for the methods of this invention are enriched for CD86^{high} or CD80^{high} expression.

In another embodiment, the dendritic cells used in the methods of this invention are selected for their capacity to expand antigen-specific CD25+CD4+ suppressor cells. In one embodiment, the DCs are isolated from progenitors or from blood for this purpose. In another embodiment, dendritic cells expressing high amounts of DEC-205/CD205 are used for this purpose.

T suppressor cell expansion, in one embodiment, is antigen-specific. In one embodiment, antigenic peptide or protein is supplied in the culture simultaneously with dendritic cell contact with CD25+ CD4+ cells. In another embodiment, dendritic cells, which have already processed antigen are contacted with the CD25+ CD4+ T cells.

In one embodiment, the term “contacting a target cell” refers herein to both direct and indirect exposure of cell to the indicated item In one embodiment, contact of CD25+ CD4+ cells to an antigenic peptide, protein, cytokine, growth factor, dendritic cell, or combination thereof, is direct or indirect. In one embodiment, contacting a cell may comprise direct injection of the cell through any means well known in the art, such as microinjection. It is also envisaged, in another embodiment, that supply to the cell is indirect, such as via provision in a culture medium that surrounds the cell, or administration to a subject, via any route well known in the art, and as described hereinbelow.

Methods for priming dendritic cells with antigen are well known to one skilled in the art, and may be effected, as described for example Hsu et al., Nature Med. 2:52-58 (1996); or Steinman et al. International application PCT/US93/03141. Antigens may, in one embodiment, be chosen for a particular application, or, in another embodiment, in accordance with the methods of this invention, as described further hereinbelow, and may be associated, in other embodiments, with fungal, bacterial, parasitic, viral, tumor, inflammatory, or autoimmune (i.e., self antigens) diseases.

In one embodiment, antigenic peptide or protein is added to a culture of dendritic cells prior to contact of the dendritic cells with CD 25+ CD4+ T cells. In one embodiment, soluble peptide or protein antigens are used at a concentration of between 10 pM to about 10 μM. In one embodiment, 30-100 ng ml⁻¹ is used. The dendritic cells are, in one embodiment, cultured in the presence of the antigen for a sufficient time to allow for uptake and presentation, prior to, or in another embodiment, concurrent with culture with CD 25+ CD4+ T cells. In another embodiment, the antigenic peptide or protein is administered to the subject, and, in another embodiment, is targeted to the dendritic cell, wherein uptake occurs in vivo, for methods as described hereinbelow

Antigenic protein or peptide uptake and processing, in one embodiment, can occur within 24 hours, or in another embodiment, longer periods of time may be necessary, such as, for example, up to and including 4 days or, in another embodiment, shorter periods of time may be necessary, such as, for example, about 1-2 hour periods.

In one embodiment, CD25+ CD4+ T cell expansion may be stimulated by a dendritic cell to T cell ratio of 1:1 to 1:10. In one embodiment, about 5 million T cells are administered to a subject.

In another embodiment, the T suppressor cells for DC expansion are enriched within a cell population by the use of marker selection. In one embodiment, the T suppressor cell population is selected for being dendritic-cell responsive, and is enriched prior to expansion for CD25^{high} expression. In one embodiment, following enrichment of a cell population for T cells expressing markers associated with a suppressor cell phenotype, such cells are then contacted with dendritic cells, and expanded in culture, as described. In another embodiment, CD8+ T suppressor cells may be expanded via the methods of this invention, wherein CD25+ CD8+ T cells are contacted with dendritic cells and an antigenic peptide or protein, and expanded in culture as described hereinabove.

In another embodiment, the CD25+ CD4+ T suppressor cells expanded by the dendritic cells in the methods of this invention are autologous, syngeneic or allogeneic, with respect to the dendritic cells. In another embodiment, the CD25+ CD4+ T suppressor cells expanded by the dendritic cells in the methods of this invention are enriched for CTLA-4high and/or GITRhigh expression. In another embodiment, the CD25+ CD4+ T suppressor cells expanded by the dendritic cells in the methods of this invention are engineered to express CTLA-4 and/or GITR.

In another embodiment, the dendritic cells used in the methods of this invention are isolated from a subject suffering from an autoimmune disease or disorder, and in another embodiment, the antigenic peptide or antigenic protein is associated with the autoimmune disease or disorder. The autoimmune disease or disorder may be any of those desrcribed hereinabove, such as for example type I diabetes, and in another embodiment, the antigenic peptide or protein may be expressed on pancreatic β cells. In one embodiment, the antigenic peptide may be a BDC mimetope. In another embodiment, the antigenic peptide or protein may be derived insulin, proinsulin, preproinsulin, islet associated antigen (IAA), glutamic acid decarboxylase (GAD), or islet-specific glucose 6 phsophatse catalytic subunit related protein (IGRP). As described hereinabove, peptide libraries from these antigens or cells producing same may be utilized for any application in this invention.

In another embodiment, the dendritic cells used in the methods of this invention are isolated from a subject with an inappropriate or undesirable inflammatory response, and in another embodiment, the antigenic peptide or protein is associated with the inappropriate or undesirable inflammatory response.

In another embodiment, the dendritic cells used in the methods of this invention are isolated from a subject with an allergic response, and in another embodiment, the antigenic peptide or protein is associated with the allergic response.

In another embodiment, the dendritic cells used in the methods of this invention are isolated from a subject who is a recipient of a transplant. In one embodiment, the dendritic cells are isolated from a donor providing a transplant to said subject, and in another embodiment, the antigenic peptide or protein is associated with an immune response in the subject receiving a transplant from a donor.

In another embodiment, the immune response is a result of graft versus host disease. In another embodiment, the immune response is a result of host versus graft disease.

In one embodiment, the DC expanded CD25+ CD4+ T cells can be used to suppress an inflammatory response, in a disease-specific manner. In one embodiment, the T suppressor cells of this invention may suppress any autoimmune disease, allergic condition, transplant rejection, or chronic inflammation due to external causes, such as, for example inflammatory bowel disease. It is to be understood that any immune response, wherein it is desired to suppress such a response, the T suppressor cells of this invention may be thus utilized, and is an embodiment of this invention.

In another embodiment the CD25+ CD4+ T suppressor cells may be expanded via the use of an agent that cross-links a T cell receptor on the T cells, which, in another embodiment, may be an antibody, which specifically recognizes CD3.

In another embodiment, the methods of this invention for expanding CD25+ CD4+ T suppressor cells may further comprise the step of culturing previously isolated, expanded CD25+ CD4+ T cells with additional dendritic cells, and the antigenic peptide, protein or agent that cross-links a T cell receptor on the T cells, for a period of time resulting in further CD25+ CD4+ T cell expansion.

In another embodiment, this invention provides a method for delaying onset, reducing incidence, suppressing or preventing autoimmunity in a subject, comprising the steps of contacting in a culture CD25+ CD4+ T cells with dendritic cells and an antigenic peptide or an antigenic protein associated with an autoimmune response in a subject, or a derivative thereof for a period of time resulting in CD25+ CD4+ T cell expansion and administering the expanded CD25+ CD4+ T cells thus obtained in to the subject, wherein the isolated, expanded CD25+ CD4+ T cells suppress an autoimmune response in the subject, thereby delaying onset, reducing incidence, suppressing or preventing autoimmunity.

In one embodiment, the culturing of CD25+ CD4+ T cells with the dendritic cells result in the enhanced functionality of the CD25+ CD4+ T cells, which in one embodiment, results in enhanced suppressive activity by the CD25+ CD4+ T cells. In one embodiment, dendritic cells instruct CD25+ CD4+ T cells to acquire functions, which lead to disease suppression. Such instruction may, in one embodiment, be over a period of time in culture, or, in another embodiment, may occur rapidly.

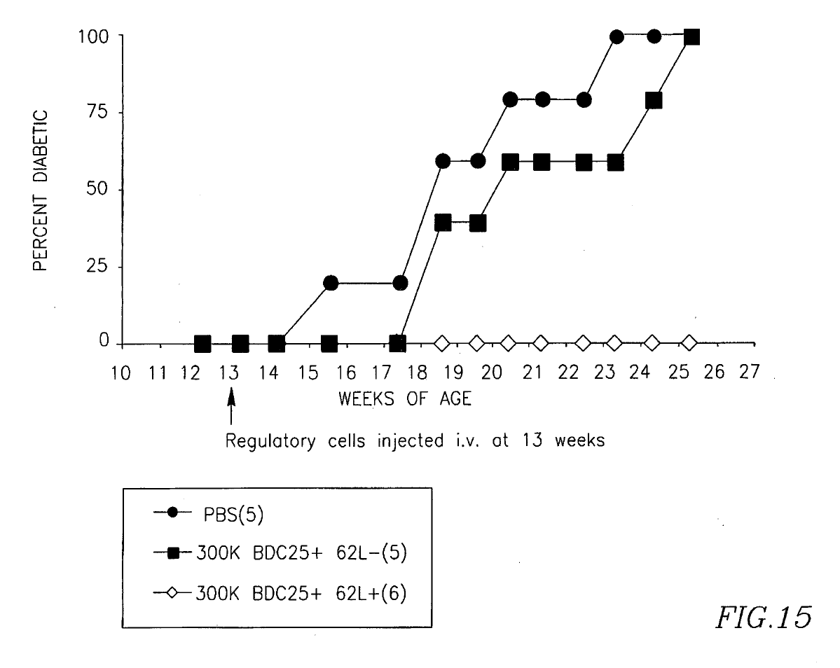

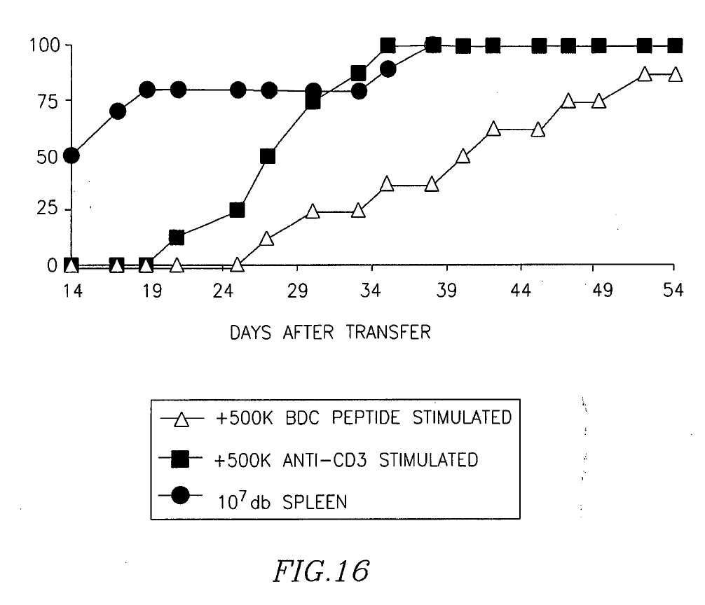

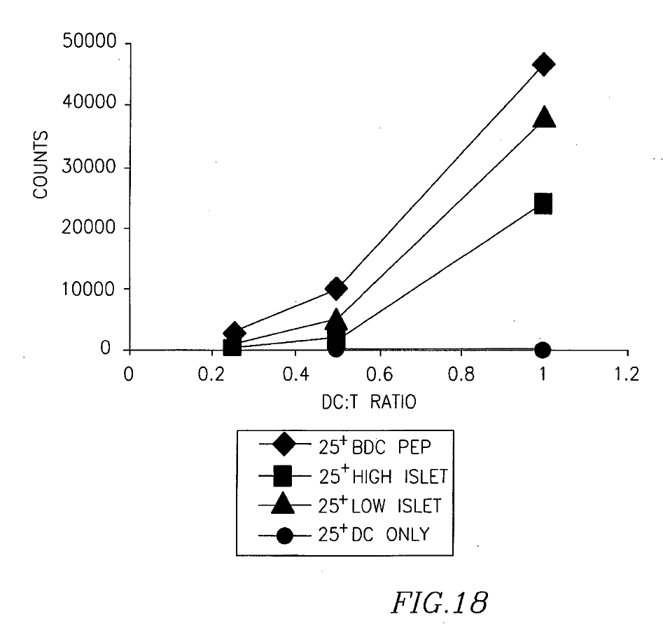

In one embodiment, expression of T suppressor cells delay autoimmunity, or in another embodiment, prevent autoimmunity, even at early stages of the disease. CD4+CD25+CD62L− cells (FIG. 15), while not preventing diabetes, nonetheless delayed its initial occurrence, whereas the CD62L+ population significantly inhibited disease, at a time where islet inflammation has progressed. In one embodiment, antigen-specific T suppressor cells prevent or significantly delay autoimmunity (FIG. 16). In one embodiment DCs process antigen by phagocytosis of diseased cells, or in another embodiment, by phagocytosis of cells which express an autoantigen, such as for example as described herein, in FIG. 18.

In one embodiment, the culturing of CD25+ CD4+ T cells with the dendritic cells is in the presence of a cytokine or growth factor, as described hereinabove.

In one embodiment, the autoimmune response results in the development of type I diabetes, and in another embodiment, the antigenic peptide or protein is expressed in pancreatic β cells. In another embodiment, the antigenic peptide is a BDC mimetope.

The injection of spleen cells from diabetic NOD mice into NOD.scid mice produces diabetes, which is mediated by T cells with a diverse repertoire of T cell receptor specificities. Injection with DC-expanded CD25+ CD4+ T suppressor cells with the diabetic spleen cells prevented diabetes development (FIG. 13).

In one embodiment of this invention, the method for delaying onset, reducing incidence or suppressing an autoimmune response in a subject is in a subject suffering from an autoimmune response directed against multiple autoantigens. In one embodiment, the CD25+ CD4+ T cells are mono-antigen specific, and according to this aspect of the invention, and in one embodiment, the mono-antigen specific CD25+ CD4+ T cells delay onset, reduce incidence or suppress an autoimmune response in the subject.

In one embodiment, the autoimmune response is a relapsing and remitting response, and in another embodiment, the CD25+ CD4+ T cells are administered to the subject during the relapsing or remitting phase of said immune response, or combination thereof.

In another embodiment, this invention provides a method for delaying onset, reducing incidence or suppressing an autoimmune response in a subject, comprising the steps of culturing an isolated dendritic cell population with an antigenic peptide or an antigenic protein associated with an autoimmune response in a subject administering the dendritic cells to a subject, whereby the dendritic cells contact CD25+ CD4+ T cells, resulting in CD25+ CD4+ T cell expansion in the subject wherein expanded CD25+ CD4+ T cells suppress an autoimmune response in the subject, thereby delaying onset, reducing incidence or suppressing an autoimmune response.

In another embodiment, administration of the cells for the methods of this invention may be in combination with traditional therapies, or in another embodiment, with reduced dosages of such traditional therapies. For example, in methods of treating, etc., autoimmunity, the methods of this invention may be accompanied by the administration of immunosuppressants, where delay or abrogation of disease is greater, or in another embodiment, wherein the dosage of the immunosuppressant is reduced, or the number of immunosuppressants administered. In one embodiment, the methods are used for treating autoimmune diabetes, and are in another embodiment, combined with insulin therapy, wherein the subject is administered insulin less frequently, or in another embodiment, at lower doses, or in another embodiment, GLP1 is administered, or in another embodiment, any agent found to ameliorate effects of the disease, whereby such administration in conjunction with the cells and/or compositions of this invention are in any way beneficial to the subject.

In one embodiment, cells for administration to a subject in this invention may be provided in a composition. These compositions may, in one embodiment, be administered parenterally or intravenously. The compositions for administration may be, in one embodiment, sterile solutions, or in other embodiments, aqueous or non-aqueous, suspensions or emulsions. In one embodiment, the compositions may comprise propylene glycol, polyethylene glycol, injectable organic esters, for example ethyl oleate, or cyclodextrins. In another embodiment, compositions may also comprise wetting, emulsifying and/or dispersing agents. In another embodiment, the compositions may also comprise sterile water or any other sterile injectable medium. In another embodiment, the compositions may comprise adjuvants, which are well known to a person skilled in the art (for example, vitamin C, antioxidant agents, etc.) for some of the methods as described herein, wherein stimulation of an immune response is desired, as described further hereinbelow.

In one embodiment, the cells or compositions of this invention may be administered to a subject via injection. In one embodiment, injection may be via any means known in the art, and may include, for example, intra-lymphoidal, or subcutaneous injection.

In another embodiment, the T suppressor cells and dendritic cells for administration in this invention may express adhesion molecules for targeting to particular sites. In one embodiment, T suppressor cell and/or dendritic cells may be engineered to express desired molecules, or, in another embodiment, may be stimulated to express the same. In one embodiment, the DC cells for administration in this invention may further express chemokine receptors, in addition to adhesion molecules, and in another embodiment, expression of the same may serve to attract the DC to secondary lymphoid organs for priming. In another embodiment, targeting of DCs to these sites may be accomplished via injecting the DCs directly to secondary lympoid organs through intralymphatic or intranodal injection.

In another embodiment, this invention provides a method for delaying onset, reducing incidence or suppressing an autoimmune response in a subject, comprising the step of contacting a dendritic cell population in vivo with an antigenic peptide or protein associated with an autoimmune response in the subject for a period of time whereby the dendritic cells contact CD25+ CD4+ T cells in said subject, stimulating antigen-specific expansion of said CD25+ CD4+ T cells in said subject, wherein expanded CD25+ CD4+ T cells suppress an autoimmune response in the subject, thereby delaying onset, reducing incidence or otherwise suppressing an autoimmune response.

In one embodiment, expression of T suppressor cells delay autoimmunity, or in another embodiment, prevent autoimmunity, even at early stages of the disease CD4+CD25+CD62L− cells (FIG. 15), while not preventing diabetes, nonetheless delayed its initial occurrence, whereas the CD62L+ population significantly inhibited disease, at a time where islet inflammation has progressed.

In one embodiment, the antigen is delivered to dendritic cells in vivo in the steady state, which, in another embodiment, leads to expansion of disease specific suppressors. Antigen delivery in the steady state can be accomplished, in one embodiment, as described (Bonifaz, et al. (2002) Journal of Experimental Medicine 196: 1627-1638; Manavalan et al. (2003) Transpl Immunol. 11: 245-58).

In one embodiment, the antigens are targeted to dendritic cells in vivo to modulate suppressor cells. In one embodiment, antigens are targeted to subsets of dendritic cells, which expand suppressors in vivo. In one embodiment, the antigen may be genetically engineered, for example, and in another embodiment, an islet cell autoantigen is engineered to be expressed as a fusion protein, with an antibody that targets dendritic cells, such as, for example, the DEC-205 antibody. Methods for accomplishing this are known in the art, and may be, for example, as described, Hawiger D. et al. J. Exp. Med., Volume 194, (2001) 769-780.

In another embodiment, select types of dendritic cells in vivo function to expand the T suppressor cells. In one embodiment, the use of dendritic cells and one antigen, will block a disease, which is caused by an autoimmune response directed to multiple antigens.

In another embodiment, dendritic cell contact with the CD25+ CD4+ T cells results in enhanced dendritic cell longevity, antigen persistence, or combination thereof. According to this aspect of the invention, and in one embodiment, the dendritic cells following contact with CD25+ CD4+ T suppressor cells may further contact CD25− T effector cells, which may, in one embodiment, be CD4+ or CD8+. In another embodiment, dendritic cells having contacted CD25+ T cells may stimulate their conversion to CD25+ expressing cells. In another embodiment, dendritic cell contact with CD25− T cells stimulates their expansion, which, in another embodiment, stimulates enhanced expansion of CD25+ T cells. In one embodiment, expansion of CD25− T cells, according to this aspect, stimulates production of a cytokine or growth factor, which, in another embodiment, may play a role in CD25+ T cell expansion.

In one embodiment, the autoimmune response is directed against multiple autoantigens, and in another embodiment, the antigen-specific expansion of CD25+ CD4+ T cells in the subject is following dendritic cell contact with a single antigen of multiple autoantigens associated with the autoimmune response.

In another embodiment, this invention provides a method for downmodulating an immune response in a subject, comprising the steps contacting in a culture CD25+ CD4+ T cells with dendritic cells and an antigenic peptide or an antigenic protein associated with an immune response in a subject, for a period of time resulting in CD25+ CD4+ T cell expansion and administering the expanded CD25+ CD4+ T cells thus obtained to a subject, wherein the isolated, expanded CD25+ CD4+ T cells downmodulate an immune response in the subject.

In one embodiment, this invention provides a method for downmodulating an immune response, which is an inappropriate or undesirable inflammatory response. In another embodiment, the immune response is an allergic response.

In another embodiment, the immune response is directed against multiple antigens, and in another embodiment, the CD25+ CD4+ T cells are mono-antigen specific, as described hereinabove.

In one embodiment, the multiple antigen source may comprise tissue itself (e.g., pancreatic islets), cell lines (e.g., beta cell lines), beta cells derived from different types of stem cells, or any other source wherein tolerance to an antigen which may be derived from that source is desired. In one embodiment, the dendritic cells may take up and process multiple antigens from complex antigen sources such as cells.

In another embodiment, the immune response is a result of graft versus host disease. According to this aspect of the invention, and in one embodiment, the dendritic cells are isolated from a donor supplying a graft to said subject. In another embodiment, the CD25+ CD4+ T cells are isolated from a donor supplying a graft to said subject. In another embodiment, the CD25+ CD4+ T cells are syngeneic or allogeneic, with respect to the dendritic cells and the subject.

In another embodiment, the immune response is a result of host versus graft disease, and in another embodiment, the dendritic cells, or in another embodiment, the CD25+ CD4+ T cells are isolated from the subject. In another embodiment, the CD25+ CD4+ T cells are syngeneic or allogeneic, with respect to the dendritic cells. In another embodiment, the antigenic peptide or protein is derived from the graft.

In one embodiment, the suppressor T cells of this invention may be administered to a recipient contemporaneously with a graft or transplant. In another embodiment, the suppressor T cells of this invention may be administered prior to the administration of the transplant. In one embodiment, the suppressor T cells of this inveniton may be administered to the recipient about 3 to 7 days before transplantation of the donor tissue. The dosage of the suppressor T cells varies within wide limits and will, of course be fitted to the individual requirements in each particular case, and may be, in another embodiment, a reflection of the weight and condition of the recipient, the number of or frequency of administrations, and other variables known to those of skill in the art. The suppressor T cells can be administered, in other embodiments, by a route, which is suitable for the tissue, organ or cells to be transplanted. The T suppressor cells of this invention may be administered systemically, i.e., parenterally, by intravenous injection or targeted to a particular tissue or organ, such as bone marrow. The suppressor T cells of this invention may, in another embodiment, be administered via a subcutaneous implantation of cells or by injection of stem cell into connective tissue, for example muscle.

In another embodiment, this invention provides a method for downmodulating an immune response, which is directed to infection with a pathogen, and the immune response is not protective to the subject.

In one embodiment, the pathogen may mimic the subject, and initiate an autoimmune repsonse. In another embodiment, infection with the pathogen results in inflammation, which damages the host. In one embodiment, the response result in inflammatory bowel disease, or in another embodiment, gastritis, which may be a result, in another embodiment, of H. pylori infection.

In another embodiment, the immune response results in a cytokine profile, which is not beneficial to the host. In one embodiment, the cytokine profile exacerbates disease. In one embodiment, a Th2 response is initiated when a Th1 response is beneficial to the host, such as for example, in lepromatous leprosy. In another embodiment, a Th1 response is initiated, and persists in the subject, such as for example, responses to the egg antigen is schistosomiasis.

According to this aspect, and in one embodiment, administration of the culture-expanded, CD25+ CD4+ T suppressor cells downmodulates the immune response, which is not beneficial to the host. In another embodiment, the method may further comprise the step of administering an agent to said subject, which elicits a cytokine profile in said subject associated with protection from said pathogen. In one embodiment, a desired cytokine profile is initiated by administration of a particular initiator cytokine, such as for example, administration of IL-12, or IFN-γ, in subjects where a Th1 response is desired.

In another embodiment, this invention provides a method for downmodulating an immune response in a subject, comprising the steps of culturing an isolated dendritic cell population with an antigenic peptide or an antigenic protein associated with an immune response in a subject and administering the dendritic cells to a subject, whereby the dendritic cells contact CD25+ CD4+ T cells, resulting in CD25+ CD4+ T cell expansion in the subject, wherein expanded CD25+ CD4+ T cells downmodulate an immune response in the subject.

In one embodiment, the term “downmodulating” refers to inhibition, suppression or prevention of a particular immune response. In one embodiment, downmodulating results in diminished cytokine expression, which provides for diminished immune responses, or their prevention In another embodiment, downmodulation results in the production of specific cytokines which have a suppressive activity on immune responses, or, in another embodiment, inflammatory responses in particular.

In one embodiment, according to this aspect of the invention, dendritic cell contact with the CD25+ CD4+ T cells results in enhanced dendritic cell longevity, antigen persistence, or combination thereof. In another embodiment, the dendritic cells contact CD25−T cell populations in said subject, resulting in antigen-specific CD25− T cell proliferation. In another embodiment, the antigen-specific CD25− T cells are memory T cells.

Antigen targeted to dendritic cells in vivo persisted for prolonged periods of time (FIG. 16). In one embodiment, this invention provides a method for modulating an immune response in a subject, comprising the steps of contacting a dendritic cell population in vivo with an antigenic peptide or protein associated with an immune response whose modulation is desired, whereby the dendritic cell population contacts CD25+ CD4+ T cells in the subject, wherein CD25+ CD4+ T cell contact promotes antigen persistence in the dendritic cell population in vivo and the dendritic cell population with persistent antigen contacts effector T cells in said subject, wherein the effector T cells modulate an immune response associated with said antigenic protein or peptide, thereby modulating an immune response in a subject.

In one embodiment, the term “modulating” refers to stimulating, enhancing or altering the immune response. In one embodiment, the term “enhancing an immune response” refers to any improvement in an immune response that has already been mounted by a mammal. In another embodiment, the term “stimulating an immune response” refers to the initiation of an immune response against an antigen of interest in a mammal in which an immune response against the antigen of interest has not already been initiated. It is to be understood that reference to modulation of the immune response may, in another embodiment, involve both the humoral and cell-mediated arms of the immune system, which is accompanied by the presence of Th2 and Th1 T helper cells, respectively, or in another embodiment, each arm individually. For further discussion of immune responses, see, e.g., Abbas et al. Cellular and Molecular Immunology, 3rd Ed., W. B. Saunders Co., Philadelphia, Pa. (1997).

In another embodiment, modulation of the immune response may result in the eliciting a “Th1” response, in a disease where a so-called “Th2” type response has developed, when the development of a so-called “Th1” type response is beneficial to the subject. One example would be in leprosy, where the antigen stimulates a Th1 cytokine shift, resulting in tuberculoid leprosy, as opposed to lepromatous leprosy, a much more severe form of the disease, associated with Th2 type responses.

In one embodiment, the term “Th2 type response” refers to a pattern of cytokine expression, elicited by T helper cells as part of the adaptive immune response, which support the development of a robust antibody response. Typically Th2 type responses are beneficial in helminth infections in a subject, for example. Typically Th2 type responses are recognized by the production of interleukin-4 or interleukin 10, for example, or IL-3, IL-5, IL-6, IL-9, IL-13, GM-CSF and/or low levels of TNF-α.

As used herein, the term “Th1 type response” refers to a pattern of cytokine expression, elicited by T Helper cells as part of the adaptive immune response, which support the development of robust cell-mediated immunity. Typically Th1 type responses are beneficial in intracellular infections in a subject, for example. Typically Th1 type responses are recognized by the production of interleukin-2 or interferon γ, IL-3, TNF-β, GM-CSF, TNF-α, and/or chemokines, such as MIP-1α, MIP-1 β, and RANTES.

In one embodiment, the reverse occurs, where a Th1 type response has developed, when Th2 type responses provide a more beneficial outcome to a subject, wherein modulation of the immune response may be accomplished via providing a shift to the more beneficial cytokine profile.

Modulation of an immune response can be determined, in one embodiment, by measuring changes or enhancements in production of specific cytokines and/or chemokines for either or both arms of the immune system. In one embodiment, modulation of the immune response resulting in the stimulation or enhancement of the humoral immune response, may be reflected by an increase in IL-6, which can be determined by any number of means well known in the art, such as, for example, by ELISA or RIA. In another embodiment, modulation of the immune response resulting in the stimulation or enhancement of the cell-mediated immune response, may be reflected by an increase in IFN-γ or IL-12, or both, which may be similarly determined.

In one embodiment, stimulating, enhancing or altering the immune response is associated with a change in cytokine profile. In another embodiment stimulating, enhancing or altering said immune response is associated with a change in cytokine expression. Such changes may be readily measured by any number of means well known in the art, including as described herein, ELISA, RIA, Western Blot analysis, Northern blot analysis, PCR analysis, RNase protection assays, and others.

In one embodiment, according to this aspect of the invention, the immune response is directed against an antigenic peptide or protein associated with infection. In one embodiment, the infection is a latent infection. In another embodiment, the immune response is not protective to the subject, or in another embodiment, comprises a cytokine profile that exacerbates disease.

In another embodiment, the methods for modulating immune responses in a subject of this invention may further comprise the step of administering an agent to the subject, which elicits a cytokine profile in the subject associated with protection from said pathogen. In one embodiment, the immune response prevents infection in the subject. In another embodiment, the immune response prevents latent infection in the subject.

Examples of infectious virus to which stimulation of an immune response according to the methods of this invention may be applicable include: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g. influenza viruses); Bungaviridae (e.g., Hantaan viruses, bunga viruses, phleboviruses and Nairo viruses); Arena viridae (hemorrhagic fever viruses); Reoviridae (erg., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes viruses'); Poxviridae (variola viruses, vaccinia viruses, pox viruses); and Iridoviridae (e.g. African swine fever virus); and unclassified viruses (e.g., the etiological agents of Spongiform encephalopathies, the agent of delta hepatities (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

Examples of infectious bacteria to which stimulation of an immune response according to the methods of this invention may be applicable include: Helicobacter pylori, Borellia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Chlamidia sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Actinomyces israelli and Francisella tularensis.

Examples of infectious fungi to which stimulation of an immune response according to the methods of this invention may be applicable include: Cryptococcus neoformans, Histoplasma capsulatum, Coccidioides immitis, Blastomyces dermatitidis,Chlamydia trachomatis, Candida albicans. Other infectious organisms (i.e., protists) include: Plasmodium sp., Leishmania sp., Schistosoma sp. and Toxoplasma sp.

In another embodiment, the immune response inhibits disease progression in said subject, or in another embodiment, the immune response inhibits or prevents neoplastic transformation in the subject.

In one embodiment, inhibition or prevention of neoplastic transformation according to the methods of this invention may be effected via the use of tumor specific antigens, such as, for example, the presence of mutated proteins which are expressed as a result of a neoplastic, or preneoplastic event. In one embodiment, the antigen is a molecule associated with malignant tumor cells, such as, for example altered ras. Non-limiting examples of tumors for which tumor specific antigens have been identified include melanoma, B cell lymphoma, uterine or cervical cancer

In one embodiment, a melanoma antigen such as the human melanoma specific antigen gp75 antigen may be used, or, in another embodiment, in cervical cancer, papilloma virus antigens may be used for the methods of this invention. Tumor specific idiotypic protein derived from B cell lymphomas, or in another embodiment, antigenic peptide or protein is derived from the Epstein-Barr virus, which causes lymphomas may be used, as well.

In another embodiment, the antigenic peptide or protein is derived from HER2/neu or chorio-embryonic antigen (CEA) for suppression/inhibition of cancers of the breast, ovary, pancreas, colon, prostate, and lung, which express these antigens. Similarly, mucin-type antigens such as MUC-1 can be used against various carcinomas; the MAGE, BAGE, and Mart-1 antigens can be used against melanomas. In one embodiment, the methods may be tailored to a specific cancer patient, such that the choice of antigenic peptide or protein is based on which antigen(s) are expressed in the patient's cancer cells, which may be predetermined by, in other embodiments, surgical biopsy or blood cell sample followed by immunohistochemistry.

The following non-limiting examples may help to illustrate some embodiments of the invention.

### EXAMPLES

### Example 1

### Dendritic Cells Stimulate CD25+ CD4+ T Cell Proliferation In Vitro

Materials and Methods

Mice

BALB/C and C57BL/6 mice were purchased from Taconic Farms (Germantown, N.Y.). OVA-specific, MHC class II restricted, TCR transgenic mice were DO11.10 (H-2d from Dr. P. Marrack) and OT-II (H-2b from Dr. F. Carbone). C57BL/6, CD80−/− CD86−/− and IL-2−/− mice were from Jackson, and BALB/C IL-2−/− mice from Drs. Maria and Juan Lafaille (New York University). Specific pathogen free mice of both sexes were used at 6-12 wks of age according to institutional guidelines.

Antibodies and Reagents

Monoclonal Abs for MHC class II (M5/114, TIB120), B220 (RA3-6B2, TIB146), CD8 (3-155, TIB211), CD4 (GK1.5, TIB207), CD3 (145-2C11, CRL1975) and HSA (J11d, TIB183) were from American Type Culture Collection (Manassas, Va.). FITC conjugated anti-CD25 (7D4), I-Ad (AMS-32), Gr1 (RB6-8C5), CD11c (HL3) and CD4 (H129.19), PE-anti-CD8a (53-6.7), B220 (RA3-6B2), CD86 (GL1), and CTLA-4 (UC10-4F10-11), biotinylated anti-CD25 (7D4), I-Ab (AF6-120.1), I-Ad (AMS-32) and mouse anti-human Vβ8 (BV8), APC-anti-CD11c (HL3), CD62L (MEL-14), CD25 (PC61) and CD4 (RM4-5), PE-streptavidin, Cychrome-streptavidin and PerCP streptavidin were from BD Bioscience PharMingen (San Diego, Calif.). FITC- and biotin-KJ1.26 antibody to the TCR of DO11.10 T cells was from Caltag (Burlingame, Calif.). Purified antibody to CD3 (145-2C11), CD25 (PC61), CD49b/Pan NK cells (DX5), CD16/CD32 (2.4G2) and control rat IgG were from BD Bioscience PharMingen. Biotin goat anti-GITR and IFN-γ was purchased from R&D systems (Minneapolis, Minn.); rHu IL-2 from Chiron (Emeryville, Calif.); anti-CD11c, CD43, CD19, CD5, FITC and PE microbeads from Miltenyi Biotec (Gladbach, Germany); carboxyfluorescein diacetate succinimidyl ester (CFSE) from Molecular Probes (Eugene, Oreg.), and intracellular staining kit for CTLA-4 and OptEIATM kits for mouse IL-2, 4, 10 and IFN-γ ELISA (BD Bioscience PharMingen).

Proliferation Assays

Spleen and lymph node cell suspensions were depleted of J11d+, CD8+ and DX5+ cells by panning. The remaining CD4+ enriched cells were stained with antibodies to CD4 and CD25 (7D4) and sorted on a FACS Vantage (BD Bioscience) into CD25+ and CD25− populations (>97% and >99% pure). 1×10⁴ T cells were cultured 3 d with APCs, either 10³ to 10⁴ DCs or 5-10×10⁴ fresh spleen cells (irradiated with 15-20 Gy) in 96 well round bottomed plates (Corning, N.Y.). 1 mg/ml OVA protein was pulsed into the bone marrow cultures for 16 hrs prior to harvesting the DCs, or 1 μg/ml DO11.10 OVA 323-336 peptide was added continuously to the APC-T cell cocultures. To assess suppression by CD25+ CD4+ T cells, whole spleen cells (5-10'10⁴) were used to stimulate mixtures of 1-2×10⁴ CD25− and 1-2×10⁴ CD25+ CD4+ T cells from DO11.10 or BALB/C mice (14-16, 21). 5% v/v supernatant of 2C11 hybridoma cells secreting anti-CD3 antibody, or 1 μg/ml purified antibody, was added for stimulation 3H-thymidine uptake (NEN; 1 μCi/well) by proliferating lymphocytes was measured at 60-72 h. To assess the need for cell-cell contact, CFSE labeled T cells were placed on both sides of a transwell chamber (Costar, Rochester N.Y.). The outer well contained DCs and T cells (3×10⁵ each) and anti-CD3 antibody to stimulate cell growth, while the inner well had 5×10⁴ T cells without or with either anti-CD3 or 5×10⁴ DCs, to determine if soluble factors from the outer well could drive T cell expansion.

Bone Marrow Derived DCs (BM-DCs)

These were prepared with GM-CSF (28). Briefly, bone marrow cells were grown in RPMI 1640 containing 5% FCS and the supernatant (3% vol/vol) from J558L cells transduced with murine GM-CSF (from Dr. A. Lanzavecchia, Basel Institute, Basel, Switzerland). On day 5, OVA (Seikagaku, Japan), which contained <20 pg endotoxin/mg protein, was added in some wells at 1 mg/ml with or without lipopolysaccharide (LPS; Sigma, St.Louis, Mo.) at 50 ng/ml for 16 h. On day 6, cells were collected and washed with HBSS. After Fc block, the cells were stained with FITC-anti-GR1 mAb and PE-anti-CD86. After washing, the cells were incubated with anti-FITC-microbeads and put onto MACS columns (Miltenyi) to eliminate residual Gr1+ granulocytes. The negative cells were then incubated with anti-PE-MACS beads and put onto MACS columns to provide CD86high mature and CD86low immature DCs, which were irradiated with 15-20 Gy; in some experiments the CD86 high and low DCs were sorted by flow cytometry with similar results. For fixation, DCs were incubated with 0.75% paraformaldebyde for 30 min on ice. To measure IL-2 production, fixed or non-fixed DCs were cultured 1 day with 0, 10, 100 or 1000 ng/ml LPS and the concentration of IL-2 measured by ELISA.

Other APCs

Spleen CD8− and CD8+ DCs were prepared as described (Iyoda, T., S. et al., 2002. J. Exp. Med. 195:1289-1302.). Splenic B cells were prepared with CD19+ MACS beads from spleen high density populations. Peritoneal exudate cells (PECs) were collected by washing the peritoneal cavity with PBS. 4d earlier, some mice were given thioglycollate (TGC; Difco, Detroit, Mich.). In some instances, 2 days after injection of TCG, mice were given 100 U IFN-γ i.p. to upregulate MHC class II on the macrophages. Lymph node CD11c+ DCs were isolated with CD11c beads (Iyoda et al., supra). For priming with Complete Freund's Adjuvant (CFA; Difco), a 1:1 emulsion of CFA and PBS was injected s.c. (50 ul/paw), and 5d later, lymph node CD11c+ DCs were prepared.

Proliferation of CFSE-Labeled CD25+ and CD25− CD4+ T Cells

For in vitro studies, FACS purified CD25+ or CD25− CD4+ T cells were incubated with 1 μM CFSE for 10 min at 37° C. and 104 T cells were cultured with OVA-pulsed or unpulsed CD86+ BM-DCs for 3 days prior to FACS analysis for proliferation (progressive halving of the CFSE label). Dead cells were gated out with TOPRO-3 iodide (Molecular Probes) labeling. For in vivo proliferation, CD25+ or CD25− CD4+ T cells purified by flow cytometry or by MACS were labeled with 5 μM CFSE, and 0.7-1.0×106 T cells were injected i.v. into BALB/c recipients. One day later, 2×105 OVA-pulsed or unpulsed, LPS-matured marrow DCs (depleted of macrophages by adherence to plastic for 2 h) was injected s.c. in each paw. Alternatively, the mice were given 25 μg of soluble endotoxin free OVA into the paw. It is known that DCs in the steady state are the major cell type presenting OVA to T cells in the steady state.

Results

CD25+ and CD25− CD4+ T cells were purified from ovalbumin (OVA) specific TCR transgenic DO11.10 mice in order to follow their antigen-dependent growth, and were evaluated by fluorescence activated cell sorting (FIG. 1A, top). This step was the limiting for the experiments, because only 2-3×10⁶ purified CD25+ CD4+ T cells were obtained from 8 mice. When standard bulk populations of spleen cells were tested as APCs, it was found, as expected that the CD25+ CD4+ T cells were anergic or non-responsive to stimulation with anti-CD3 mitogenic antibody, whereas the CD25− CD4+ T cells responded (FIG. 1A, bottom). Furthermore, mixtures of CD25+ and CD25− CD4+ T cells were suppressed, failing to proliferate to anti-CD3 when spleen cells were the APCs (FIG. 1A). In contrast, when DCs (even in low numbers) generated from bone marrow progenitors with granulocyte macrophage colony stimulating factor (GM-CSF) were tested, the CD25+ CD4+ T cells were now responsive to anti-CD3, and suppression was no longer evident in mixtures of CD25+ and CD25− CD4+ T cells (FIG. 1A). Strong responses were repeatedly observed with CD25+ CD4+ T cells from two different OVA-specific transgenics, DO11.10 and OT-II, and over a broad range of DC doses in the presence of OVA antigen (FIG. 1B). Non-TCR transgenic BALB/C T cells also responded to DCs presenting anti-CD3 but did not respond to DCs presenting OVA, while DO11.10 T cells responded to both (FIG. 1C), confirming that the responses by OVA-reactive, CD25+ CD4+, transgenic T cells were antigen-specific.

To evaluate the effect of DC maturation on their capacity to stimulate CD25+ CD4+ T cells, the bone marrow-derived DCs were sorted into mature and immature populations, expressing high and low levels of the CD86 T cell costimulatory molecule respectively (FIG. 1D). Both were active, but the mature CD86^{high} DCs were better stimulators for T cell proliferation when either OVA protein or preprocessed peptide was the source of antigen (FIG. 1E). Dose response studies indicated that as little as 0.01 μg/ml peptide could stimulate the proliferation of CD25+ CD4+ T cells significantly. Also, the DCs were equally active if they had been matured spontaneously (FIG. 1D) or in the presence of lipopolysaccharide (LPS), the latter to increase the yield of mature DCs. Therefore CD25+ CD4+ T cells are not intrinsically unresponsive to TCR stimulation but are able to proliferate to anti-CD3 and to antigen when presented by DCs and in the absence of exogenous growth factors like IL-2.

To certify the capacity of CD25+ CD4+ T cells to proliferate to antigen presenting DCs, their growth was documented in two other ways. First, the number of CD25+ CD4+ cells expanded about 5 fold in 3-5 days in the presence of OVA antigen (FIG. 2A, right), at the same time that DNA synthesis was robust, 50-100×10³ cpm in cultures of 10⁴ T cells (FIG. 2A, left). However, the CD25+ CD4+ T cells did not expand beyond the initial 3-5 days of culture, whereas CD25− CD4+ cells expanded in a sustained fashion (FIG. 2A), the latter most likely because of the production of large amounts of IL-2 as will be shown below. Stimulation of another 2-3 fold expansion by rechallenging the CD25+ CD4+ T cells with additional antigen-bearing DCs was also verified following one week in culture.

Proliferation of CFSE-labeled CD25+ CD4+ and CD25− CD4+ T cells was then compared. Both populations underwent several cycles of cell division in 3 days (FIG. 2B). Using this data and the approach of Wells et al (Wells, A. D. et al., 1997. J. Clin. Invest. 100: 3137-3183), it was found in 6 experiments (3 each using DCs to present anti-CD3 antibody or specific OVA antigen), that about one-third of the cultured CD25+ CD4+ T cells underwent at least one mitotic event during 3 days of culture (FIG. 2D). During the same time period, a similar frequency of the CD25− CD4+ T cells entered cell cycle, but the number of mitotic events was actually less (FIG. 2D). The major CD62L+ and minor CD62L− subsets of CD25+ CD4+ T cells were found to respond comparably to DC-OVA. Therefore, in the first 3 days of culture, both CD25+ CD4+ and CD25− CD4+ were stimulated by DCs to enter cell cycle and to expand significantly.

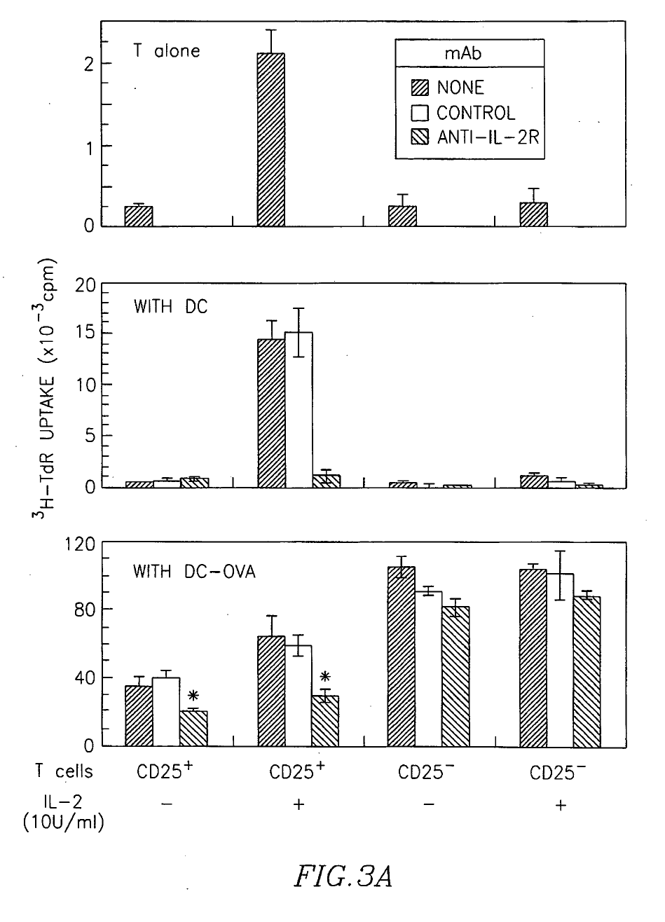

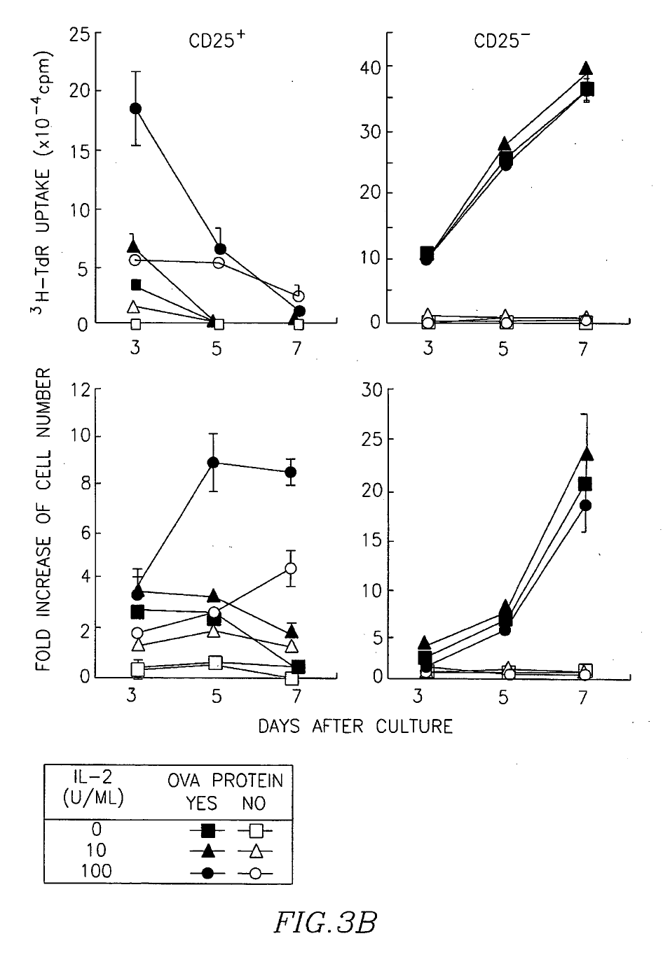

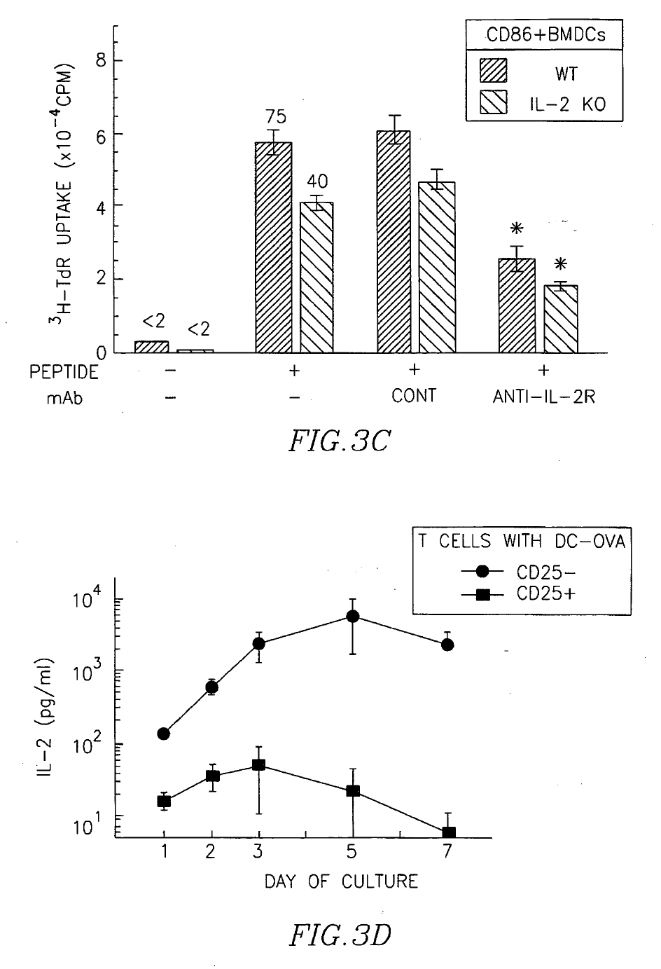

Since the CD25 marker for regulatory T cells is a component of the IL-2 receptor, the role of IL-2 in these cultures was tested. The addition of exogenous IL-2 only induced a minute response in the CD25+ CD4+ T cells themselves (FIG. 3A, top; note the units on the y-axis). However, IL-2 did induce more significant proliferation of CD25+ CD4+ T cells (but not CD25− CD4+ T cells) in the presence of DCs without OVA antigen; this increase in responses could be blocked by anti-IL-2R antibody completely (FIG. 3A, middle). DCs with OVA stimulated CD25+ CD4+ T cell growth 5-10 fold more vigorously than in the absence of antigen (compare the y-axes of FIG. 3A, middle and bottom). The response of CD25+ CD4+ T cells was enhanced by low doses of exogenous IL-2 (FIG. 3A). Proliferation in the absence of IL-2 was partially blocked (52.0±9.3%, n=5) by anti-CD25 antibody, whereas IL-2 and anti-IL-2R antibody had little or no effect on the responses of CD25− CD4+ T cells (FIG. 3A, bottom). When the kinetics of the response to exogenous IL-2 was monitored, the stimulation of CD25+ CD4+ T cell growth was evident primarily in the first 3-5 days in culture (FIG. 3B, left). In contrast, CD25− CD4+ T cells responded continuously for one week to DCs, without any boost by exogenous IL-2 (FIG. 3B, right). Thus IL-2 enhanced antigen-dependent and independent proliferation of CD25+ CD4+ T cells in response to DCs.

### Example 2

### Dendritic Cells Stimulated CD25+ CD4+ T Cell Proliferation is Partially Dependent Upon Dendritic Cell B7 Expression

To determine whether the observed proliferative responses to DCs could be attributed to IL-2 made by the DCs themselves, DCs from IL-2−/− mice and aldehyde-fixed DCs were utilized. DCs in the absence of T cells produced IL-2 upon stimulation, which could be abolished by fixation of the DCs in paraformaldehyde. DCs from IL-2−/− mice (FIG. 3C) were active in stimulating CD25+ CD4+ T cells, and the growth was partially blocked with anti-CD25 antibody (FIG. 3C). IL-2 production in the IL-2−/− DC-T cell cocultures was then measured, since it is known that CD25+ CD4+ T cells do not produce detectable IL-2 in response to splenic APCs and anti-CD3. However, culture supernatants from CD25+ CD4+ T cells and OVA-DCs from wild type mice did contain some IL-2 by ELISA (concentrations of IL-2 above the bars in FIG. 3C), but primarily in the first 3 days of the cultures and only at a small fraction of the levels induced by DCs from CD25− CD4+ T cells (FIG. 3D). IL-10 was undetectable by ELISA in the culture supernatants of CD25+ T cells stimulated by DC-OVA (<40 pg/ml), and other cytokines like IFN-γ (<40 pg/ml) and IL-4 (<10 pg/ml) were also absent (ELISA).

In order to assess the potential role of cell surface costimulators on DCs, formaldehyde fixed DC induction of T cell proliferation was determined. Live DCs were more effective than fixed DCs (FIG. 4A, 3 fold higher doses of DCs were used; FIG. 4B). Titrated anti-IL-2R Ab again could not block the proliferation of CD25+ CD4+ T cells completely in both live and fixed DCs (FIG. 4A). Nevertheless, aldehyde-fixed DCs stimulated the growth of CD25+ CD4+ and CD25− CD4+ T cells in the presence of OVA antigen. In the absence of OVA but with IL-2, live and fixed DCs also stimulated the growth of some CD25+ CD4+, but not CD25− CD4+, T cells (FIG. 4B).

The activity of aldehyde fixed DCs suggested that a membrane bound costimulatory molecule was contributing to the T cell response. In fact, DCs prepared from mice genetically deleted of the CD80 and CD86 costimulatory molecules (also known as B7-1 and B7-2) were only ⅓ as efficient at stimulating the proliferation of CD25+ CD4+ cells (FIG. 4C). The proliferation of the transgenic CD25− CD4+ T cells in parallel was actually maintained with B7-deficient DCs in this system in which the DC:T cell ratio was 1:1 (FIG. 4C), but B7-deficient DCs were less active with lower DC:T cell ratios of 1:25. In sum, the response of CD25+ CD4+ T cells to antigen bearing DCs was substantially blocked by anti-CD25 antibody The requisite IL-2 was produced in small amounts by the responding T cells, and B7 costimulation contributed significantly to CD25+ CD4+ T cell proliferation.

### Example 3

### Dendritic Cell Stimulated, Culture-Expanded CD25+ CD4+ T Cells Maintain Phenotype and Function

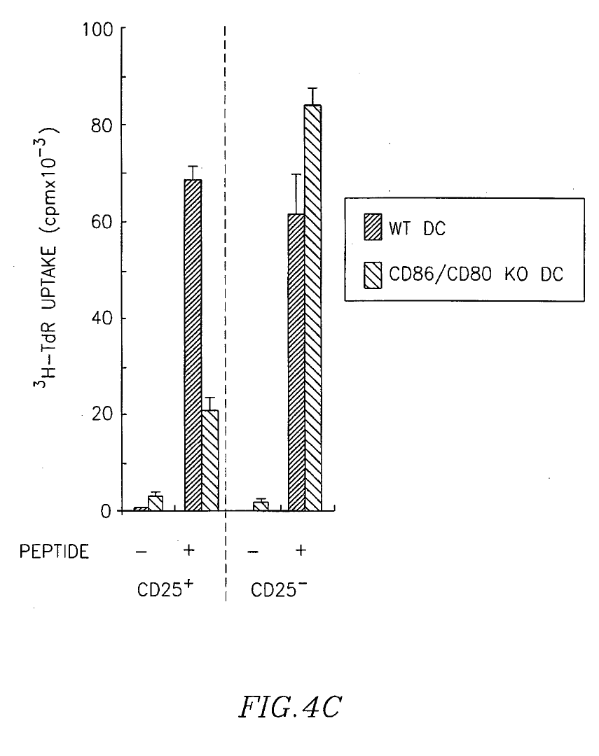

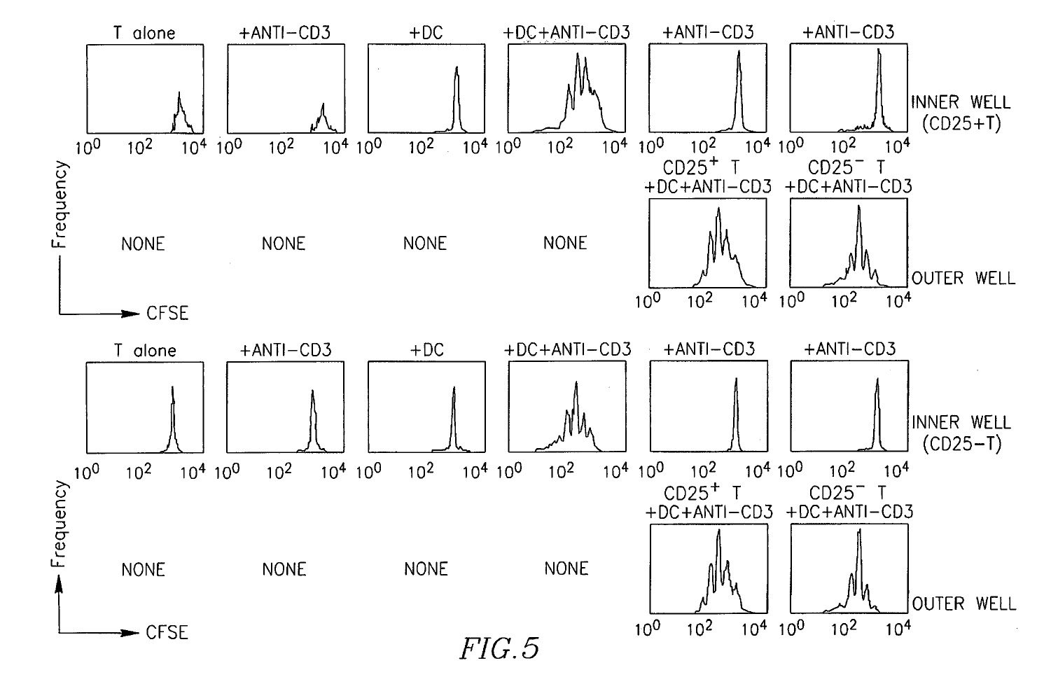

Transwell experiments were then carried out to determine whether proliferation of CD25+ CD4+ T cells induced by DCs requires DC-T cell contact. These T cells, when cultured in the inner well with anti-CD3 or with DC only, could undergo at most a single cell division whether or not the outer well was empty or contained mixtures of CD25+ CD4+ T cells with both DC and anti-CD3 antibody (FIG. 5, top). However, most CD25+ CD4+ T cells cultured together with DCs and anti-CD3 divided 2-5 times (FIG. 5), indicating that cell-cell contact with DCs was important for initiating their growth.

It was important to verify that the CD25+ CD4+ T cells retained their known phenotypic markers and suppressive properties following their DC-induced expansion, 3-10 fold in the absence and presence of exogenous IL-2 respectively. In terms of phenotype, the expanded CD25+ CD4+ T cells maintained higher expression of CTLA-4 and GITR relative to CD25− CD4+ responders (FIG. 6A). During expansion, expression of CD62L (the lymph node horning receptor) decreased on many of the CD25+ CD4+ T cells, but after 7 d of culture, most cells expressed CD62L, as is the case for most regulatory T cells in lymphoid organs. CD25− CD4+ T cells proliferating in response to DC-OVA upregulated expression of CD25, CTLA-4 and GITR, and almost all cells had little or no CD62L at day 7 (FIG. 6A). The percentage of CD25+ CD4+ T cells expressing the KJ1.26 clonotypic TCR marker was enriched following expansion, 80% vs. 60% initially, and the mean fluorescence for KJ1.26 expression increased slightly (FIG. 6B), indicating that DC-OVA were selectively expanding OVA-specific cells.

When the functions of the expanded CD25+ CD4+ cells were tested with whole spleen APCs, the T cells were indeed anergic upon challenge with OVA or anti-CD3 (FIG. 6C, groups 2 and 5 respectively) in contrast to the robust responses of CD25− CD4+ cells (FIG. 6C, groups 1 and 4). Furthermore, the expanded CD25+ CD4+ cells could actively suppress the responses of CD25− CD4+ cells to OVA or anti-CD3 (FIG. 6C, groups 3, 6 and 8). The CD25+ CD4+ T cells expanded by DC-OVA were more active on a per cell basis than freshly isolated CD25+ CD4+ T cells when tested for their capacity to suppress OVA-specific T cell responses (FIG. 6D). These findings on the retained phenotype and function of CD25+ CD4+ T cells also were noted following expansion with DC-OVA plus IL-2 (FIG. 6D, bottom). In summary, following expansion by DCs, CD25+ CD4+ T cells expressed their characteristic markers and regulatory function.

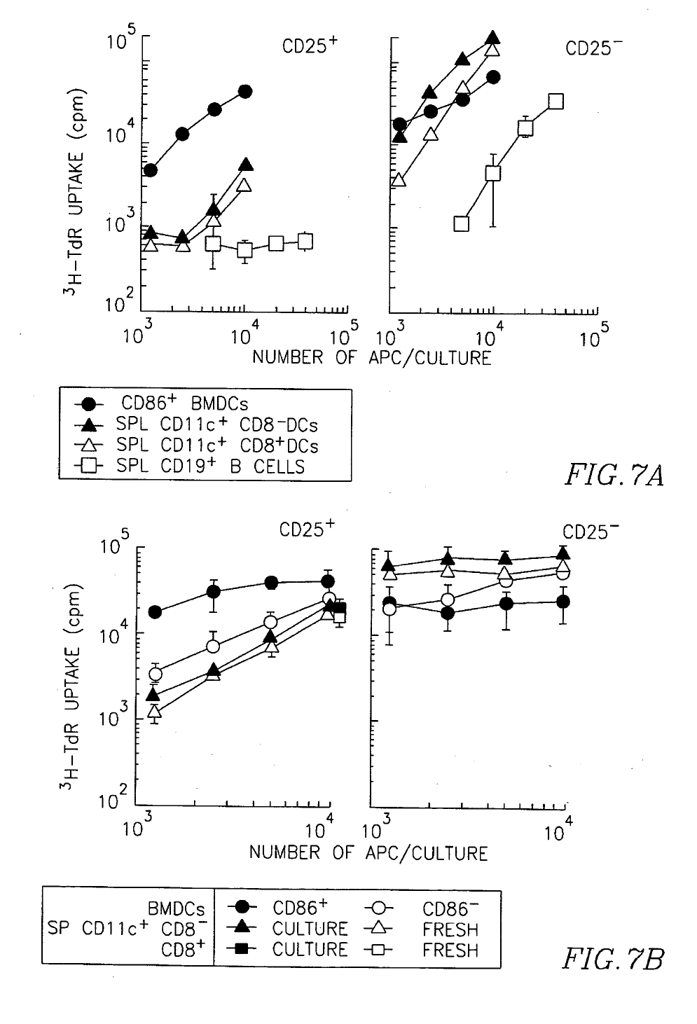

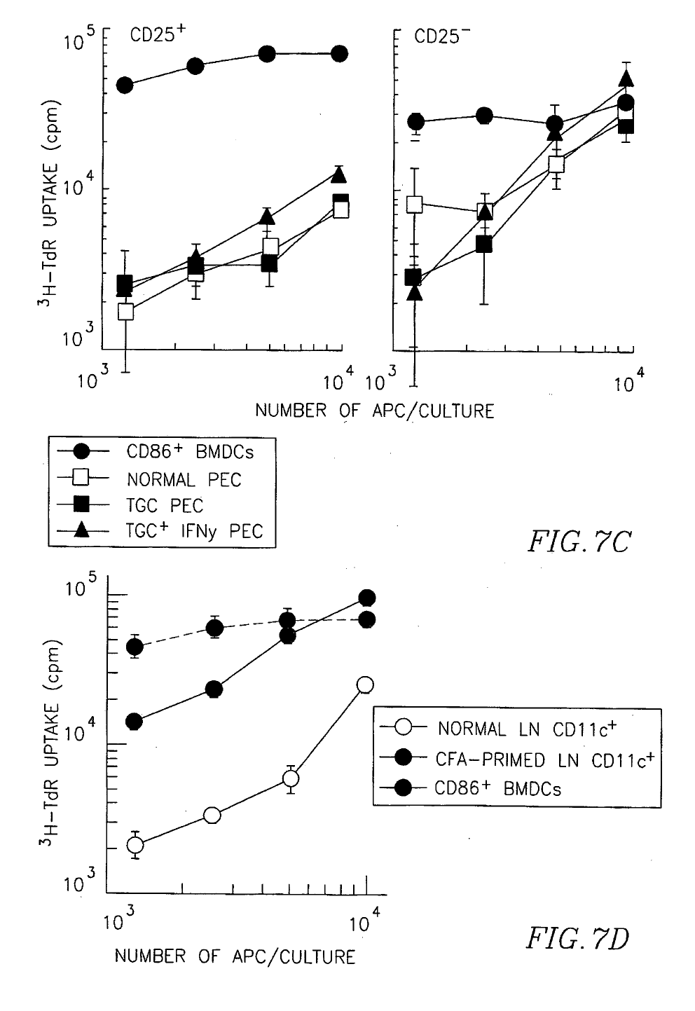

To compare the responses of CD25+ CD4+ T cells to various sources of APCs, DCs from different sites were examined. Splenic CD8+ and CD8− DC subsets tested immediately upon isolation or following maturation overnight with LPS, could stimulate CD25+ CD4+ T cells but to a much lesser degree than bone marrow DCs with either OVA protein or peptide as antigen (FIGS. 7A,B). The cultured splenic DCs had similar surface levels of CD80 and CD86 to the bone marrow DCs, but were much weaker APCs for CD25+ CD4+ T cells. However, both splenic and marrow-derived DCs were comparably potent in stimulating CD25− CD4+ T cells (FIG. 7A). CD19+ B cells stimulated with LPS overnight could elicit some T cell proliferative responses from CD25− CD4+ but not from CD25+ CD4+ T cells (FIG. 7A). Normal and thioglycollate elicited peritoneal macrophages were weak stimulators of both CD25+ and CD25− CD4+ T cells, even when the macrophages were taken from mice given IFN-γ i.p. to enhance expression of antigen presenting MHC class II products (FIG. 7C). Since bone marrow derived DCs were generated in the presence of the inflammatory cytokine GM-CSF and in the presence of other phagocytes like neutrophils and macrophages, DCs from lymph nodes expanded in the presence of an in vivo inflammatory stimulus, complete Freund's adjuvant (CFA) was tested. The CD11c+ DCs from CFA stimulated lymph nodes were 4 fold more numerous, and on a per cell basis, the CFA elicited lymph node DCs were stronger stimulators of the growth of CD25+ CD4+ regulatory T cells, compared to lymph node DCs in the steady state (FIG. 7D). Therefore DCs seem to be the major APC capable of stimulating the growth of CD25+ CD4+ T cells but acquire greater activity when matured under inflammatory conditions, either with GM-CSF in vitro or CFA in vivo.

### Example 4

### Adoptively Tranferred, Dendritic Cell Stimulated, Culture-Expanded CD25+ CD4+ T Cells Proliferate In Vivo

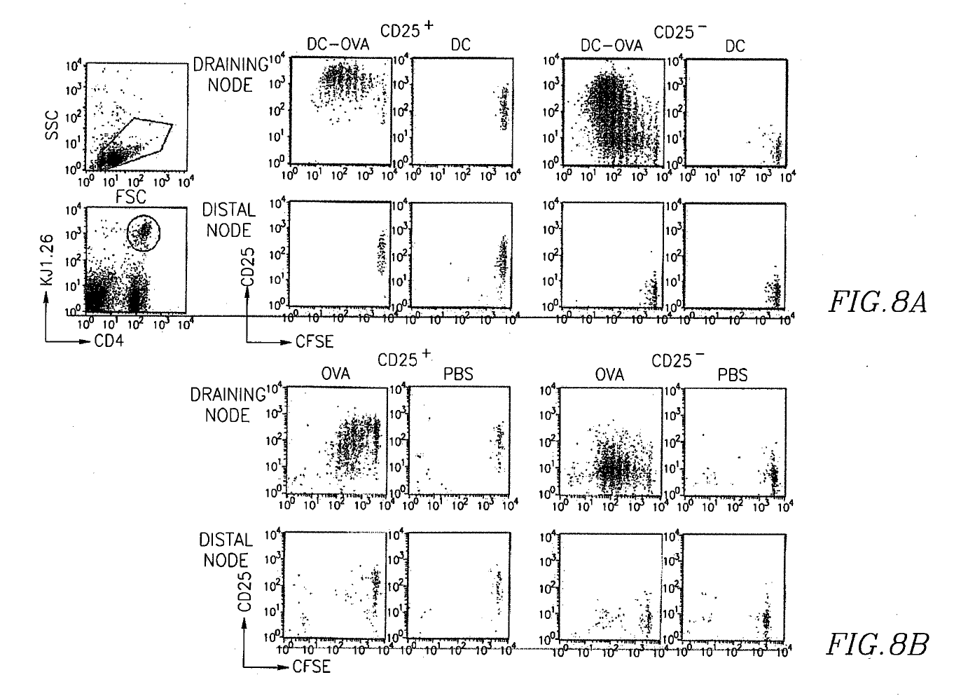

Purified CD25+ and CD25− CD4+ T cells from OVA-specific TCR transgenic mice were labeled with CFSE, injected the T cells into naive BALB/C mice, and followed their proliferation and distribution in response to challenge with OVA antigen, to extend the findings to the growth of CD25+ CD4+ T cells in vivo. In each of 3 experiments, CD25+ CD4+ T cells proliferated in the draining but not distal lymph nodes (FIG. 8A) and spleen of mice challenged with DC-OVA. DC-OVA also induced extensive proliferation of CD25− CD4+ T cells in lymph nodes draining the DC injection site (FIG. 8A). The proliferation was OVA antigen-dependent, being absent in CD25+ or CD25− CD4+ T cells when animals received DCs that had not been exposed to OVA (FIG. 8A). The total number of clonotype (KJ1.26) positive T cells recovered upon stimulation with DC-OVA vs. DC was increased 8-10 fold when either CD25+ or CD25−CD4+ T cells were stimulated in vivo. However, the absolute numbers of clonotype positive CD25+ CD4+ T cells in the lymphoid organs were always lower than expanded CD25−CD4+ T cells. Interestingly, the levels of CD25 on the expanding CD25+ CD4+ regulatory T cells were increased during their growth in vivo and much higher at day 3 than the CD25 expressed by responding CD25− CD4+ T cells. These results in mice replicate the findings in vitro that DCs are able to expand CD25+ CD4+ regulatory T cells.

To determine if DCs in vivo in the steady state could stimulate the expansion of CD25+ CD4+ T cells, the latter were adoptive transferred into mice followed by challenge with soluble OVA in the absence of any adjuvant or inflammatory stimulus. It is known that DCs are the main cell type that successfully captures and presents OVA for stimulation of T cells. Again, the adoptively transferred CD25+ CD4+ and CD25− CD4+ T cells each underwent several cycles of cell division in vivo in the draining lymph nodes in response to OVA (FIG. 8B) As in the case of proliferation stimulated by injected mature DCs, CD25+ CD4+ T cells stimulated in the steady state continued to express high levels of CD25, while their CD25− CD4+ counterparts had not yet upregulated CD25 expression at this time point (FIG. 8B). Therefore CD25+ CD4+ T cells, and not contaminants in the adoptively transferred populations, proliferate to antigen bearing DCs in the steady state and after immigration from peripheral tissues.

### Example 5

### DCs Expand CD25+ CD4+ T Cells from Autoimmune NOD Mice

Materials and Methods

Mice

NOD and NOD.scid (both I-Ag7) mice were purchased from Jackson Labs (Bar Harbor, Me.) BDC2.5 TCR transgenic mice on the NOD genetic background were provided by Drs. D. Mathis and C. Benoist, Joslin Diabetes Center, Boston, Mass. Specific pathogen free mice of both sexes were used at 5-12 wks of age according to institutional guidelines. Protocols were approved by the Institutional Animal Care and Use Committee at Rockefeller University.

Antibodies

MAbs for MHC class II (TIB120), B220 (TIB146), CD8 (TIB211), CD4 (GK1.5), CD3 (145-2C11) and HSA (J11d) were from American Type Culture Collection (Manassas, Va.). FITC-conjugated anti-CD25 (7D4), I-Ag7 (OX-6), Gr1 (RB6-8C5), CD11c (HL3), and CD4 (H129.19), CD86 (GL1), biotinylated anti-CD25 (7D4), APC-anti-CD11c (HL3), CD62L (MEL-14), CD25 (PC61), and CD4 (RM4-5), and PE-streptavidin were from BD Biosciences (San Jose, Calif.). Purified antibody to CD3 (145-2C11), CD49b/Pan NK cells (DX5), CD16/CD32 (2.4G2) and control Rat IgG were also from BD Biosciences. A hybridoma expressing the anti-clonotype antibody specific for the BDC2.5 TCR (aBDC) was generously provided by Dr. O. Kanagawa, Washington Univ., St. Louis Mo., and the antibody was purified and biotinylated.

Bone Marrow-Derived DCs

Bone marrow-derived DCs were prepared with GM-CSF as previously described (Yamazaki, S., et al., 2003. J. Exp. Med. 198:235-247; Inaba, K., et al., 1992. J. Exp. Med. 176:1693-1702). DCs were isolated from normoglycemic NOD males. On day 5, LPS (Sigma-Aldrich, St. Louis, Mo.) was added at 50 ng ml⁻¹ for approximately 16 hours. On day 6, cells were collected and the more mature Gr1- CD86+ cells were purified with FITC and PE magnetic microbeads (Miltenyi Biotec, Auburn, Calif.) as described (Yamazaki, supra) and irradiated with 15 Gy before use as antigen presenting cells.

Proliferation Assays and Expansion

Spleen and lymph node cell suspensions were enriched for CD4+ cells by panning, and sorted on a FACS Vantage (BD Biosciences, San Jose, Calif.) into CD25+ CD4+ and CD25− CD4+ populations (>95% and >97% pure) 10⁴ T cells from BDC2.5 or NOD mice were cultured for 3 days with the indicated number of DCs and a mimetope peptide (termed 1040-55; 30-100 ng ml⁻¹) having the sequence RVRPLWVRME (38), or with purified anti-CD3 antibody (0.3-1 mg ml⁻¹) Recombinant Human IL-2 (Chiron Corp, Emeryville, Calif.) was added where indicated, at a concentration of 100 U ml⁻¹. All CD25+ CD4+ T cell expansions for in vivo injection were performed with IL-2 in the cultures. To assess suppression by CD25+ CD4+ T cells, 5×10⁴ whole NOD spleen cells irradiated with 15 Gy were used to stimulate mixtures of 1×10⁴ CD25− CD4+ and the indicated number of CD25+ CD4+ T cells from BDC2.5 or NOD mice. If DC-expanded CD25+ CD4+ T cells were used, CD11c+ cells were removed using magnetic microbeads (Miltenyi Biotec, Auburn, Calif.) after harvesting the cells on day 5-7. [3H]-thymidine uptake, 1 mCi/well (Perkin Elmer, Boston, Mass.) by proliferating lymphocytes was measured at 60-72 hours.

Results

In order to demonstrate that autoantigen-specific CD25+ CD4+ T cells expand in response to DCs, autoreactive T cells that responds to a natural autoantigen and are diabetogenic, were used. CD4+ T cells from BDC2.5 TCR transgenic NOD mice respond to a protein expressed by islet β cells. Although the β cell autoantigen remains to be identified, a series of mimetope peptides have been uncovered, which stimulate proliferation of BDC2.5 T cells, one of which was as the antigen, referred to as BDC peptide. This particular mimetope peptide has a high functional affinity (low EC50) and also stimulates normal NOD T cells.

A more than 95% pure CD25+ CD4+ BDC2.5 T cell and NOD bone marrow DC cell population was isolated, the latter via using magnetic beads to enrich for CD86+ NOD DCs; which expressed high levels of CD86, comparable to other strains (FIG. 9a).

BDC2.5 CD25+ CD4+ T cell culture with NOD CD86+ DCs pulsed with BDC peptide, resulted in T cells proliferation by day 3 (FIG. 9b). Proliferation also took place in response to CD86− DCs pulsed with BDC peptide, but it was more limited. CD25− CD4+ cells likewise proliferated to DCs with BDC peptide, but the addition of IL-2 did not significantly change proliferative responses. CD25+ CD4+ T cells cultured with DCs and IL-2 (but not BDC peptide) also showed significant proliferation, as was evident with ovalbumin-specific CD25+ CD4+ T cells, in Example 1, but the combination of IL-2 and BDC peptide with DCs was most effective, resulting in higher ³H-thymidine incorporation than with CD25− CD4+ cells. Proliferation of CD25+ CD4+ T cells cultured with spleen antigen presenting cells, a TCR stimulus and IL-2 has been reported (Takahashi, T., et al., 1998. Int. Immunol 10:1969-1980; Thornton, A. M., and E. M. Shevach. 1998. J. Exp. Med. 188:287-296), however these conditions (BDC2.5 T cells, spleen antigen presenting cells, BDC peptide, and IL-2) were 3.5 fold less efficient for CD25+ CD4+ T cell proliferation (FIG. 9b). Relative to the number of cells placed into culture, there was a 5-10 fold expansion in the number of recovered T cells from cultures of CD25+ CD4+ T cells, DCs, and peptide with and without IL-2 at 5 days.

CD25+ CD4+ and CD25− CD4+ T cells expanded similarly up to day 5, but only the latter continued to expand up to day 7 (FIG. 9c). Thus CD25+ CD4+ T cells from BDC2.5 transgenic mice can grow in response to DCs in an antigen-specific manner, in much the same way as demonstrated for ovalbumin-specific T cells (Example 1).

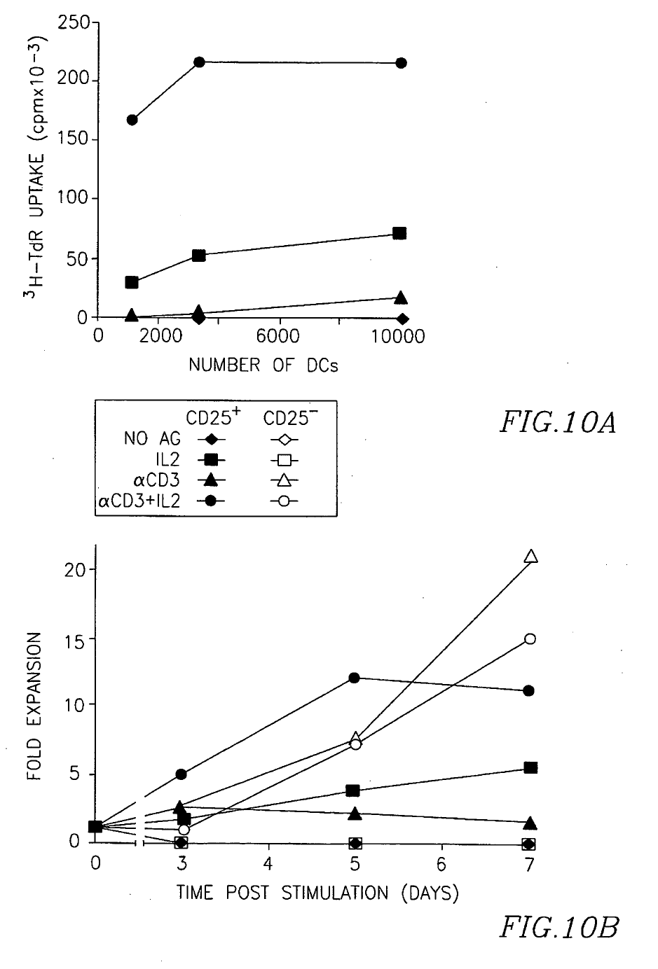

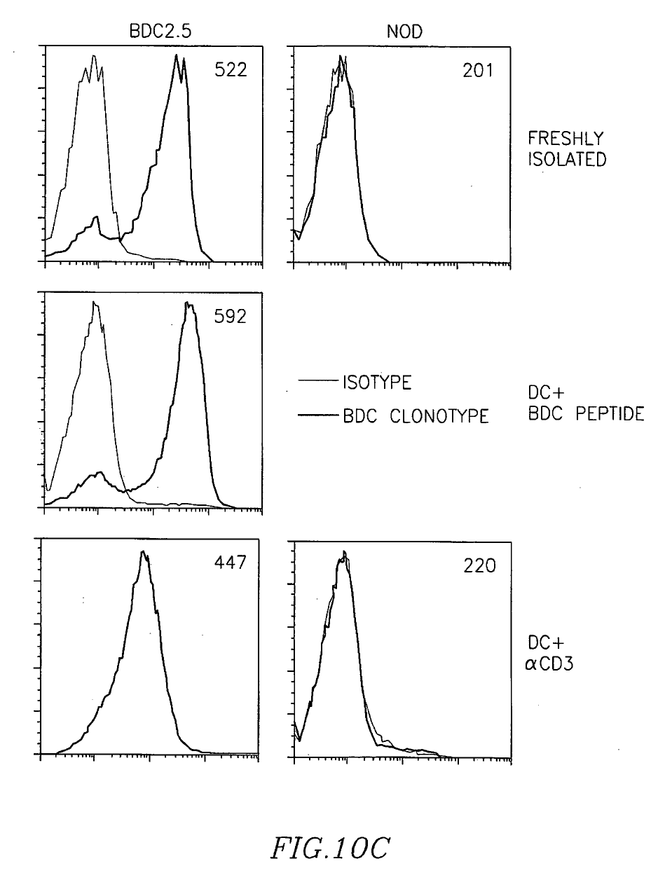

Non-transgenic, regulatory T cells from autoimmune NOD mice were also capable of proliferation and expansion with DCs. CD25+ CD4+ T cells isolated from NOD mice, and stimulated with NOD CD86+ DCs and anti-CD3, demonstrated induced DNA synthesis and proliferation (FIGS. 10a and b). The T cells also proliferated when cultured with DCs and IL-2 in the absence of a TCR stimulus, but IL-2, DCs and anti-CD3 synergized to induce very high levels of DNA synthesis and expansion of cell numbers, over 10-fold by 5 days. In contrast, NOD CD25+ CD4+ T cells cultured without DCs but with IL-2 with or without anti-CD3 gave only 2×10³ or 7×10³ cpm of DNA synthesis, respectively These results indicated that both DCs and T cells (either BDC2.5 or non-transgenic) from autoimmune NOD mice interact to significantly expand CD25+ CD4+ regulatory T cells.

While roughly 80% of freshly isolated or DC+ IL-2-expanded BDC2.5 CD25+ CD4+ T cells expressed high levels of the BDC2.5 TCR (FIG. 10c), BDC2.5 CD25+ CD4+ T cells stimulated with DCs, IL-2 and anti-CD3, demonstrated significantly diminished clonotype expression. Freshly isolated, or DC/anti-CD3 stimulated NOD CD25+ CD4+ T cells did not express significant levels of the BDC clonotype (FIG. 10c). Since T cells expressing a transgenic TCR also can express endogenous TCR α chains, expression of 2 different endogenous TCRα (Vα2 and Vα8.3) was determined, and did not change significantly after DC/peptide stimulation. Thus, DCs select and expand suppressor T cells specific for the presented TCR ligand, and in contrast to T cells expanded with DC-anti-CD3, BDC2.5 T cells expanded with DC-peptide express much higher levels of TCR on their cell surfaces.

### Example 6

### DCs Expand Antigen Specific CD25+ CD4+ T Cells In Vivo

Materials and Methods

All methods and reagents were as listed in Example 1, with CD25+ CD4+ and CD25− CD4+ cells purified by flow cytometry, and labeled with 5 mM carboxyfluorescein diacetate succinimidyl ester (CFSE; Molecular Probes, Eugene, Oreg.), and 3.3×10⁵ T cells were injected i.v. into NOD recipients. 1 day later, 2×10⁵ BDC peptide-pulsed or unpulsed, LPS-matured bone marrow DCs were injected s.c. in each paw. 3 d after DCs were injected, lymph nodes were collected, and cells were stained with CD4 and BDC2.5 clonotype antibody, and the level of CFSE staining determined by flow cytometry.

Results

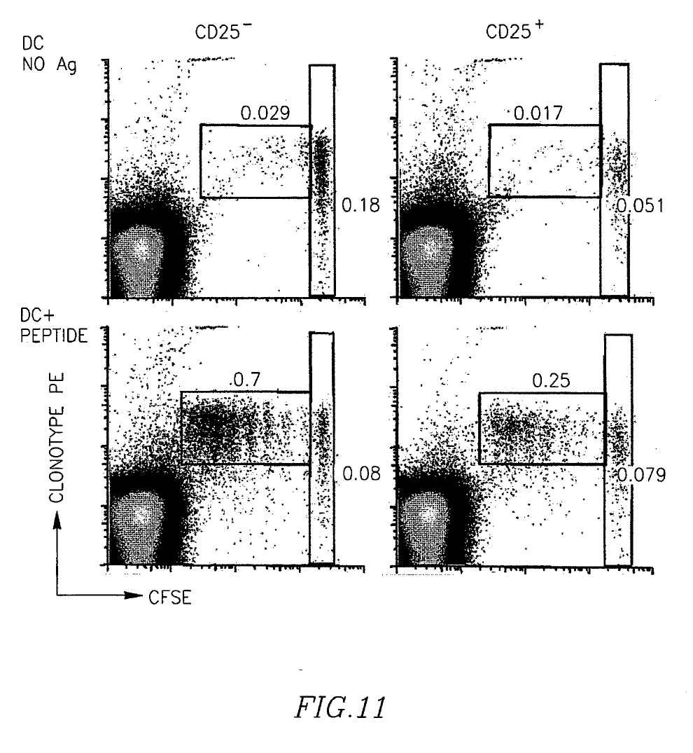

Purified CD25+ CD4+ T cells from BDC2.5 mice, were labeled with CFSE prior to injection into NOD mice, followed by s.c. injection of mature marrow derived DCs that had been pulsed (or not pulsed, serving as controls) with BDC peptide. Proliferation was assessed 3 days later, determined by progressive halving of the amount of CFSE per T cell. The CD25+ CD4+ T cells proliferated, with up to 6 divisions per cell, in the draining lymph nodes of mice that received BDC peptide-pulsed DCs but not in mice that received PBS or DCs alone (FIG. 11). A similar proliferative response was observed with control CD25− CD4+ cells, but CFSE was not diluted in either CD25+ or CD25− CD4+ cells in the distal lymph nodes (FIG. 11) Therefore, DCs are able to induce proliferation of CD25+ CD4+ T cells from an autoimmune strain in vivo.

### Example 7

### In Vivo, Dendritic Cell Stimulated, Culture-Expanded CD25+ CD4+ T Cell Suppression of Autoimmune Diabetes

Materials and Methods

Diabetes Induction

Diabetes was induced in NOD.BDC2.5 mice with one dose of cyclophosphamide (Sigma) at 200 mg/g in PBS. 3 days later, mice were injected with PBS or 5×10⁵ CD25+ CD4+ or CD25− CD4+ T cells, which had been expanded with DCs and BDC peptide in vitro for 5-7 days. In separate experiments, diabetes was transferred to NOD.scid mice with 3-10×10⁶ spleen cells (given i.v.) from female diabetic NOD mice. At the same time, the indicated numbers of purified CD25+ CD4+ or CD25− CD4+ T cells, which had been expanded with DCs, BDC peptide, and IL-2 in vitro for 5-7 days, were also given i.v. For all diabetes experiments, development of diabetes was monitored with chemstrips (Roche Applied Science, Indianapolis, Ind.), which detects urine glucose above 150 mg dL-1. A mouse was considered diabetic on the first of 3 consecutive readings of high urine glucose. Statistics were calculated using the Mann-Whitney U test.

Histological Analysis

Pancreas tissue was fixed in Bouin's solution, and paraffin-embedded sections were stained with hematoxylin and eosin. Tissue cuts were made 100 microns apart to avoid counting any islets twice. Insulitis was assessed for each islet, and scored with a: 0, which indicates no evidence of insulitis; 1, which indicates evidence of peri-insulitis; 2, which indicates evidence of less than 70% infiltration; or 3 which indicates evidence of more than 70% infiltrated.

Results

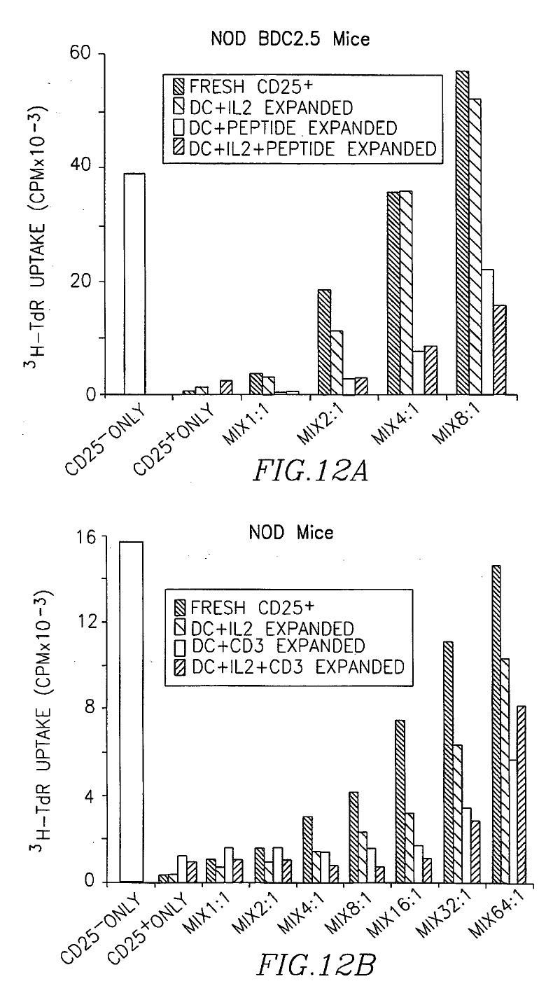

CD11c+ DCs from 7-day expansion cultures were removed, while the CD25+ CD4+ T cells were added to responder CD25− CD4+ T cells, in different ratios to measure the inhibition of CD25− CD4+ proliferation in response to BDC peptide presented by spleen APCS. Freshly isolated CD25+ CD4+ T cells, as well as CD25+ CD4+ T cells expanded with DCs and IL-2, were able to suppress, but only partially, and at high doses, i.e., when mixed 1:2 with CD25− CD4+ cells. In contrast, CD25+ CD4+ T cells expanded with peptide (without or with IL-2) had stronger activity, showing suppression even at a ratio of one CD25+ CD4+ T cell for every 8 CD25− CD4+ cells (FIG. 12a). The suppressive function of NOD CD25+ CD4+ T cells expanded with DCs and anti-CD3 was also tested. Again the T cells expanded with DCs and TCR stimulus suppressed proliferation by NOD CD25− CD4+ T cells ˜4 fold more efficiently than freshly isolated CD25+ CD4+ T cells (FIG. 12b). Whereas freshly purified NOD CD25+ CD4+ T cells showed approximately 75% suppression at a ratio of 8 responder cells for 1 CD25+ CD4+ T cell, NOD CD25+ CD4+ T cells expanded with DCs and anti-CD3 (with or without IL2) showed similar suppression at a ratio of 32:1. Therefore either polyclonal or mono-specific CD25+ CD4+ T cells from NOD mice can be expanded with DCs and anti-CD3 or antigen, and they show ˜4 fold enhancement in suppressive function.

To determine whether BDC2.5 CD25+ CD4+ T cells expanded in vitro with DCs and antigen inhibit the development of diabetes, 2 diabetes models were utilized. In the first model, suppression of pathogenic T cells of the same BDC2.5 specificity was determined. BDC2.5 mice on an NOD background did not develop diabetes spontaneously, however when young BDC2.5 NOD mice were given one injection of cyclophosphamide, diabetes developed 4-7 days later in 100% of the mice. BDC2.5.NOD mice were injected with DC-expanded CD25+ CD4+ T cells from BDC2.5 mice, 3 days post cyclophosphamide treatment, and suppression of diabetes induction was determined. In two experiments, a delay in diabetes onset, and a reduced diabetes incidence was found. In contrast, injection of DC-expanded CD25− CD4+ from BDC2.5 mice had little effect on diabetes development (FIG. 13a). Thus, DC expanded suppressor T cells were able to suppress autoimmunity even when the disease was developing rapidly.

The second diabetes model employed the injection of spleen cells from diabetic NOD mice into NOD.scid females, where autoimmune diabetes is mediated by pathogenic T cells with a diverse repertoire of T cell receptor specificities. Varied doses of DC-expanded CD25+ CD4+ T cells from BDC2.5 mice were injected with 3-8×10⁶ spleen cells from diabetic mice into NOD.scid females. The mice receiving diabetic spleen cells alone developed diabetes starting at 3-4 weeks after injection.

In the first dose response study, addition of 3×10⁵, 1×10⁵, or 3×10⁴ expanded BDC2.5 CD25+ CD4+ T cells to 3×10⁶ diabetic spleen completely prevented diabetes development (FIG. 13b). In contrast, when 3×10⁵ DC-expanded CD25− CD4+ cells were injected together with diabetic spleen cells, there was a marked acceleration of diabetes onset when compared to diabetic spleen cells alone.

In a second dose response experiment, the number of diabetic spleen cells was increased to 8×10⁶, and the number of expanded CD25+ CD4+ T cells was titrated down further. Again 50,000 DC-expanded BDC2.5 CD25+ CD4+ T cells completely prevented diabetes development. Addition of 5,000 of these regulatory cells delayed onset of diabetes, and even 500 DC-expanded BDC2.5 CD25+ CD4+ T cells demonstrated a significant delay in diabetes onset compared to those receiving spleen cells from diabetic mice alone (FIG. 13c).

The numbers of autoantigen-specific CD25+ CD4+ T cells necessary to delay or block diabetes development were much lower than the numbers of bulk (polyclonal) NOD CD25+ CD4+ T cells used in other transfer studies, i.e., 2-5×10⁵ cells were necessary to see a significant delay in diabetes development (Szanya, V., et al., 2002. J. Immunol. 169:2461-2465; Wu, Q., et al., 2001. J. Exp. Med. 193:1327-1332; Gregori, S., et al., 2003. J. Immunol. 171:4040-4047). To establish the need for antigen-specific T cells in disease suppression, and to confirm that DC stimulation alone was not sufficient for in vivo suppression, antiCD3/DC-expanded NOD CD25+ CD4+ T cells were transferred to NOD.scid mice along with spleen cells from diabetic mice. Polyclonal NOD CD25+ CD4+ T cells, even at a concentration of 10⁵, whether freshly isolated or anti-CD3/DC-expanded, provided no delay in diabetes onset (FIG. 13d). Therefore, autoantigen-specific DC-expanded CD25+ CD4+ T cells function efficiently in vivo to suppress autoimmunity mediated by autoreactive T cells.

### Example 8

### In Vivo, Dendritic Cell Stimulated, Culture-Expanded CD25+ CD4+ T Cells Suppress Autoimmune Diabetes Even After Disease Initiation

Materials and Methods

Results

Pancreata were isolated from NOD.scid mice, which still had normal glucose levels following their protection from diabetes by small numbers of BDC2.5-specific CD25+ CD4+ T cells, 80 days after transfer (FIG. 13c). Insulitis was scored from H&E sections. The mice from the groups that received 5,000 or 50,000 BDC2.5-specific CD25+ CD4+ T cells (the latter group were all diabetes free), had lymphocytic infiltrates in half of the islets scored. Representative fields from both protected groups indicated that protected mice progressed past the initiation of islet inflammation, checkpoint I, but maintained a non-destructive insulitis, and therefore were blocked at checkpoint II. The results demonstrate that protected mice have lymphocytic infiltrates in the pancreas. Pancreata from mice that did not develop diabetes by day 80 after transfer in the experiment in 5C were scored for insulitis. 150 islets from 5 mice were scored from the group which received 50,000 DC-expanded BDC CD25+ CD4+ T cells, and 48 islets from 2 mice from the group which received 5,000 cells. Representative fields for a mouse from the group which received 5000 (top) or 50,000 (bottom) suppressor T cells. Large field is 5×; inset is 20×.

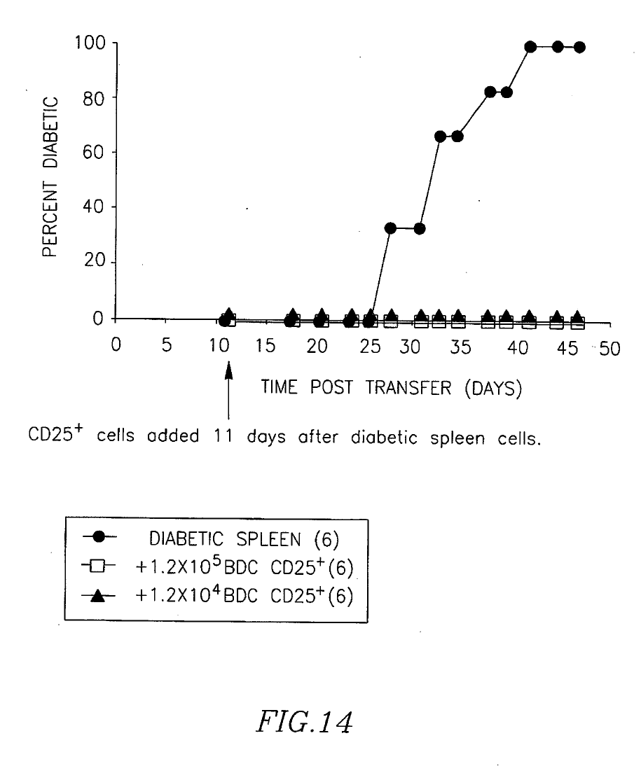

One feature of the NOD.scid system is that T cells, when injected into a lymphopoenic host, undergo antigen independent, homeostatic proliferation. To lessen the effect of such proliferation on the CD25+ CD4+ T cells, they were injected after the diabetogenic spleen cells. Even when given 11 days after the diabetogenic cells, as few as 12,000 DC-expanded BDC2.5 CD25+ CD4+ T cells prevented diabetes development (FIG. 14). Therefore, CD25+ CD4+ T cells blocked diabetes even after the diabetogenic cells have been given time to occupy the lymphoid compartments, and initiate diabetes pathogenesis.

### Example 9

### In Vivo Antigen Delivery to Dendritic Cells Results in Enhanced Antigen Persistence for Presentation to T Cells

Materials and Methods

C57Bl/6 mice were administered ovalbumin. At 1, 3, 5 and 7 days following ovalbumin administration, 1×10⁶ Ova-specific T cells labeled with CFSE, as above, are administered intravenously to the mice. 3 days following T cell transfer, lymph nodes were harvested, passed through nylon mesh to create a single cell suspension, and were analyzed by FACS for CSFE, with the dilution of the signal, representing halving of the dye, taken as a measure of T cell proliferation.

Results

The targeting of antigen, in this case ovalbumin (OVA) to dendritic cells, in vivo, resulted in CD25− CD4+ T cell expansion in vivo, in T cells specific for the antigen. The addition of OVA-specific T cells even 7 days or 15 days post-antigen delivery to DEC-205 dendritic cells, resulted in T cell expansion. Unless the antigen is targeted in vivo to DEC-205 dendritic cells, it does not persist for prolonged periods of time, to allow for effector T cell proliferation. The results demonstrated that dendritic cells can present antigen for a long time in vivo. Ovalbumin (OVA) antigen targeted to DEC 205 dendritic cells in vivo, followed by the addition of OVA-specific T cells 1, 3, 7 days or 15 days later, stimulated effector T cell proliferation, as measured by progressive halving of the amount of CFSE dye present in the sample. Ovalbumin administered alone, with anti-CD40, or dendritic cells cultured ex-vivo, and administered, failed to provide for prolonged stimulation of effector T cell proliferation.

### Example 10

### Antigen-Specific CD4+CD25+CD62L+ T Cells Inhibit Diabetes Development

Materials and Experimental Methods

13 week old NOD female mice were injected intravenously with PBS (n=5), CD4+CD25+CD62L− cells (n=5), or CD4⁺CD25⁺CD62L⁺ cells (n=6). Diabetes was monitored weekly by urine glucose.

Results

When CD4⁺CD25⁺CD62L⁺ cells suppressor T cells, expanded with DCs, were given to 13-week-old NOD mice that had not yet developed disease, disease was prevented, even at a point in time in these mice where islet inflammation has likely progressed. The CD4+CD25+CD62L− population had little if any effect on diabetes development (FIG. 15 squares). Thus islet-specific regulatory T cells are capable of blocking diabetes in both the NOD.scid transfer (as described hereinabove) and spontaneous NOD (FIG. 15) diabetes systems. In addition, further subdividing of the CD4+CD25+ T cells into a CD62L+ fraction gives greater efficacy.

### Example 10

### DC-Expanded, Islet Specific Regulatory T Cells from NOD Mice Delay Diabetes

Materials and Experimental Methods

10⁷ spleen cells from diabetic mice, ± NOD CD4+CD25+ cells stimulated with DCs and either anti-CD3 or BDC peptide were transferred to NOD.scid females. Diabetes was monitored by measuring urine glucose levels every 2-3 days

Results

In order to determine if antigen-specific regulatory cells were more efficacious in preventing diabetes, antigen-specific cells isolated from a polyclonal NOD repertoire and expanded with islet antigen plus DCs, were compared to NOD regulatory cells expanded with DCs plus a non-specific stimulus for their ability to block diabetes development. NOD CD4+CD25+CD62L+ regulatory cells expanded with DCs and IL-2±BDC peptide or anti-CD3 were transferred with 10⁷ diabetic spleen cells to NOD.scid mice. FIG. 16 demonstrates that BDC peptide-stimulated NOD regulatory cells, or antigen-specific regulatory cells significantly delayed diabetes development, as compared to non-specific anti-CD3-stimulated cells.

### Example 11

### Islet β Cells are Processed and Presented by DCs and Stimulate Suppressor T Cell Proliferation

Materials and Experimental Methods

DCs purified from bone marrow cultures and dissociated islet cells purified from NOD mice, were separately labeled with red (DCs) or green (islets) fluorescent dyes then mixed overnight at the a 1:1 and 3:1 DC: islet cell ratios and temperatures at 4° or 37° C.

DCs purified from bone marrow cultures incubated overnight with the islet cells were washed and cultured with CD4+CD25+CD62L+ T cells isolated from BDC2.5 mice, or DCs±BDC peptide, which served as controls.

Results

In order to determine whether DCs process islet antigens from islet cells, then expand disease specific suppressors from a polyclonal repertoire, DCs were loaded with islets and their presentation of islet autoantigen recognized by BDC2.5 TCR transgenic T cells was determined.

Such a scenario was of interest, for applications such as the use of the suppressors in subjects predisposed to diabetes, or those with recently developed diabetes.

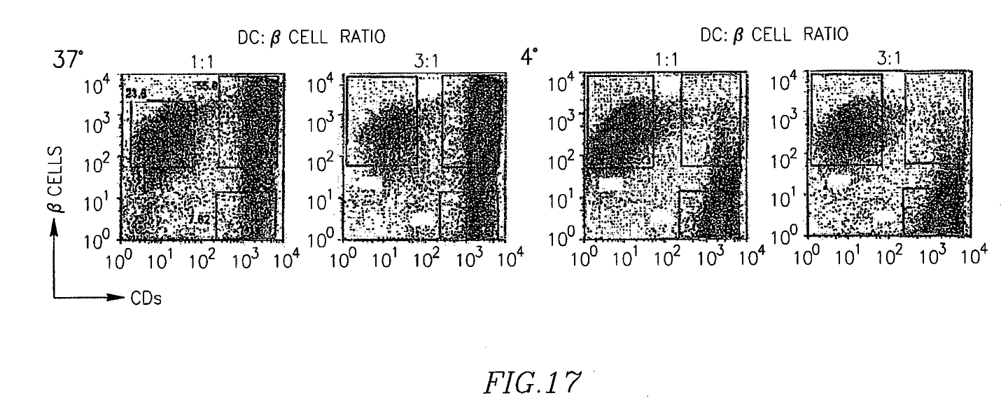

Two different experiments were performed. First, the ability of DCs to take up beta cells was shown by labeling BMDCs with one fluorescent dye and dissociated islets with another color. When analyzed by flow cytometry, double positive cells indicated DCs that had engulfed beta cells at 37° but not at 4°, as expected for active uptake (FIG. 17). This experiment illustrates the efficient capacity of DCs to take up islet cells.

Next, responses of suppressor T cells isolated from BDC2.5 mice to islet-loaded DCs were determined Suppressor T cell proliferation was observed at both islet concentrations, indicating that the DCs were capable of processing and presenting the natural BDC2.5 antigen from islets (FIG. 18).

### Example 12

### Treatment of Diabetic Mice with Islet-Specific Tregs and Limited Administration of Insulin and GLP-1

Materials and Experimental Methods

Diabetes Treatment Groups:

CD4⁺CD25⁺CD62L⁺ cells were isolated from BDC2.5 mice, and expanded for 1 week with BDC-peptide loaded DCs and IL2. Diabetic mice were identified within 5 days of onset, and then given insulin via pellets that secrete continuously for 2-3 weeks. At the same time, injections of GLP-1 were started, and continued 3 times per week for 3 weeks, in all mice. Within 3 days of the diabetic mice receiving the insulin pellet, one group of 5 mice were injected i.v. with 1.5×10⁶ DC-expanded CD25⁺ CD62L⁺ cells from BDC2.5 mice (BDC Treg), whereas another group of 4 mice received only PBS.

Glucose Tolerance:

Mice were kept without food for 15 hours then were given 2 mg/g bodyweight of glucose intraperitoneally. Blood glucose levels were monitored at intervals over a course of 200 minutes. Groups included the controls: 1-12-wk non-diabetic NOD mice (n=4), and recently diabetic mice that had undergone insulin and GLP-1 treatment, but had returned to high blood glucose (n=2), as well as mice treated with GLP-1 and DC-expanded CD25⁺ CD62L⁺ cells from BDC2.5 mice.

Histopathology:

Salivary glands and pancreata were evaluated histologically. Each islet was scored as having no insulitis (white), peri-insulitis (light grey), intra-insulitis with <60% infiltrate (dark grey), or intra-insulitis with >60% infiltrate (black).

Results

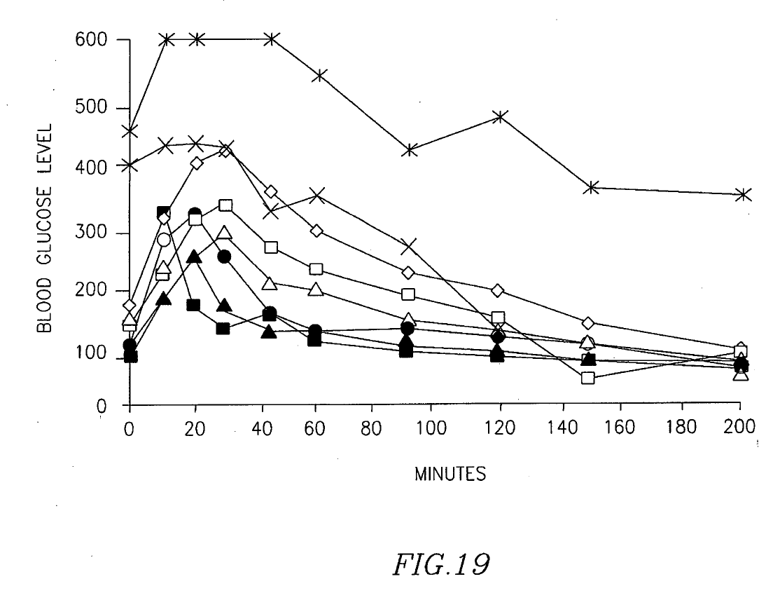

In order to determine whether suppression of diabetes could be accomplished by the administration of the T suppressor cells, reversion of overt diabetes in NOD mice treated with GLP-1 and islet-specific Tregs was evaluated. In all 4 control mice that did not receive Tregs, blood glucose levels increased soon after circulating insulin levels decreased. Three of the five mice, which had received the BDC Tregs, however, had blood glucose readings below 200, even 90 days post-treatment (FIG. 19).

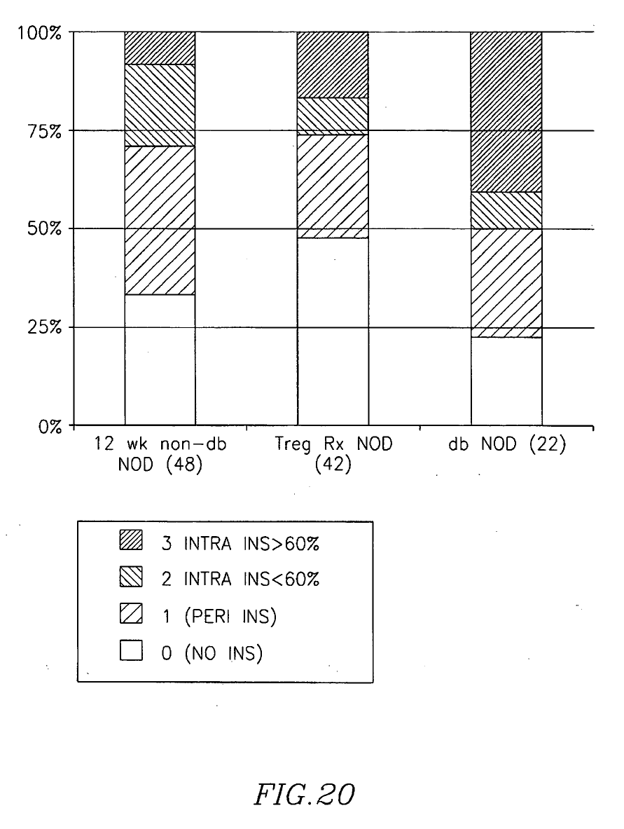

These mice were then evaluated for their glucose tolerance, in comparison to the two control groups, non-diabetic NOD mice, and recently diabetic mice that had undergone insulin and GLP-1 treatment, but had returned to high blood glucose. In non-diabetic control mice, blood glucose levels returned to normal in ˜60 minutes following challenge, whereas the circulating glucose levels in diabetic mice remained high for at least 120 minutes. Treg treated mice demonstrated glucose levels in between the 2 control groups. At 90 minutes, the average blood glucose in the nondiabetic controls was 110, in the diabetic controls was 350, and in the Treg treated mice was 190 (FIG. 20), indicating that treatment of diabetic mice with Treg helps restore glucose tolerance.

Histological evaluation of the pancreas and salivary glands of the mice demonstrated the presence of inflammation in all mice evaluated, indicating that the Treg treatment of diabetes was antigen-specific, unable to block autoimmunity to salivary gland antigens. In the diabetic control mice, few intact islets were found, and these had extensive infiltrate (FIG. 21). In the non-diabetic controls, there was a mix of uninfiltrated islets, peri-insulitis, and intra-insulitis. Mice treated with islet-specific Tregs had infiltrate similar to non-diabetic mice, with only 25% of the islets exhibiting intra-insulitis.

1. An isolated, culture-expanded T suppressor cell population, wherein said population expresses CD25 and CD4 on its cell surface.

2. The isolated culture-expanded T suppressor cell population of claim 1, wherein said population further expresses CD62L on its surface.

3. The isolated culture-expanded T suppressor cell population of claim 1, wherein said population is antigen specific.

4. (canceled)

5. The isolated culture-expanded T suppressor cell population of claim 3, wherein said antigen is a self-antigen, or a derivative thereof.

6. The isolated culture-expanded T suppressor cell population of claim 5, wherein said self antigen is expressed on pancreatic β cells.

7. The isolated culture-expanded T suppressor cell population of claim 1, wherein said population expresses a monoclonal T cell receptor.

8. The isolated culture-expanded T suppressor cell population of claim 1, wherein said population expresses polyclonal T cell receptors.

9.-14. (canceled)

15. A method for producing an isolated, culture-expanded T suppressor cell population, comprising:
a contacting CD25+ CD4+ T cells with dendritic cells and an antigenic peptide, an antigenic protein, or a derivative thereof, or an agent that cross-links a T cell receptor on said T cells in a culture, for a period of time resulting in antigen-specific CD25+ CD4+ T cell expansion; and
b. isolating the expanded CD25+ CD4+ T cells obtained in (a),
thereby producing an isolated, culture-expanded T suppressor cell population.

16. The method of claim 15, wherein said T cells are CD62L+.

17. The method of claim 15, further comprising the step of adding a cytokine to the dendritic cell, CD25+ CD4+ T cell culture.

18. The method of claim 15, wherein the cytokine is interleukin-2.

19. The method of claim 15, wherein said dendritic cells express a costimulatory molecule.

20. The method of claim 19, wherein said dendritic cells are enriched for CD86^{high} expression.

21. The method of claim 15, wherein said dendritic cells are selected for their capacity to expand antigen-specific CD25+CD4+ suppressor cells.

22. The method of claim 15, wherein said CD25+ CD4+ T cells are autologous, syngeneic or allogeneic, with respect to said dendritic cells.

23. The method of claim 15, wherein said CD25+ CD4+ T cells are enriched for CTLA-4^{high} and/or GITR^{high} expression.

24. The method of claim 15, wherein said dendritic cells are isolated from a subject suffering from an autoimmune disease or disorder.

25. The method of claim 24, wherein said antigenic peptide or antigenic protein or derivative thereof is associated with said autoimmune disease or disorder.

26. The method of claim 24, wherein said autoimmune disease or disorder is type I diabetes.

27. The method of claim 26, wherein said antigenic peptide or protein is expressed in pancreatic β cells.

28. The method of claim 27, wherein said antigenic peptide is a BDC mimetope.

29.-37. (canceled)

38. The method of claim 15, wherein said agent that cross-links a T cell receptor on said T cells is an antibody which specifically recognizes CD3.

39. The method of claim 15, wherein said expanded CD25+ CD4+ T cells are polyclonal.

40. The method of claim 15, wherein said expanded CD25+ CD4+ T cells are monoclonal.

41. The method of claim 15, further comprising the step of culturing the isolated, expanded CD25+ CD4+ T cells obtained in (c), with additional isolated, cultured dendritic cells, and said antigenic peptide, antigenic protein or derivative thereof or agent that cross-links a T cell receptor on said T cells, for a period of time resulting in further CD25+ CD4+ T cell expansion.

42. A method for delaying onset, reducing incidence, suppressing or treating autoimmunity in a subject, comprising the steps of:
a contacting in a culture CD25+ CD4+ T cells with dendritic cells and an antigenic peptide or an antigenic protein or a derivative thereof, associated with an autoimmune response in a subject, for a period of time resulting in CD25+ CD4+ T cell expansion; and
b. administering the expanded CD25+ CD4+ T cells obtained in (a) to a subject,
wherein said isolated, expanded CD25+ CD4+ T cells inhibit, suppress or prevent an autoimmune response in said subject, thereby delaying onset, reducing incidence, suppressing or treating autoimmunity.

43. The method of claim 42, wherein said dendritic cells are isolated from said subject.

44. The method of claim 42, wherein said dendritic cells express a costimulatory molecule.

45. The method of claim 44, wherein said dendritic cells are enriched for CD86^{high} expression.

46. The method of claim 42, wherein said CD25+ CD4+ T cells are isolated from said subject.

47. The method of claim 42, wherein said CD25+ CD4+ T cells are CD62L+.

48. The method of claim 42, wherein said CD25+ CD4+ T cells are syngeneic or allogeneic, with respect to said dendritic cells and said subject.

49. The method of claim 42, wherein said CD25+ CD4+ T cells are enriched for CTLA-4^{high} and/or GITR^{high} expression.

50. The method of claim 42, wherein said expanded CD25+ CD4+ T cells are polyclonal.

51. The method of claim 42, wherein said expanded CD25+ CD4+ T cells are monoclonal.

52. The method of claim 42, wherein expansion of said CD25+ CD4+ T cells is antigen specific.

53. The method of claim 42, further comprising the step of adding a cytokine in step (a).

54. The method of claim 42, wherein said antigenic peptide or protein is expressed in pancreatic β cells.

55. The method of claim 42, wherein said antigenic peptide is a BDC mimetope.

56. The method of claim 42, wherein said autoimmunity results in the development of type I diabetes.

57. The method of claim 42, wherein said autoimmunity is directed against multiple autoantigens.

58. The method of claim 57, wherein said CD25+ CD4+ T cells are mono-antigen specific.

59.-60. (canceled)

61. A method for downmodulating an immune response in a subject, comprising the steps of:
a. contacting in a culture CD25+ CD4+ T cells with dendritic cells and an antigenic peptide or an antigenic protein associated with an immune response in a subject, or a derivative thereof, for a period of time resulting in CD25+ CD4+ T cell expansion; and
b. administering the expanded CD25+ CD4+ T cells obtained in (a) to a subject,
wherein said isolated, expanded CD25+ CD4+ T cells downmodulate an immune response in said subject.

62. The method of claim 61, wherein said immune response is an inappropriate or undesirable inflammatory response.

63. The method of claim 61, wherein said immune response is an allergic response.

64. The method of claim 61, wherein said immune response is directed against multiple antigens.

65. The method of claim 64, wherein said CD25+ CD4+ T cells are mono-antigen specific.

66. The method of claim 61, wherein said immune response is a result of graft versus host disease.

67. The method of claim 66, wherein said dendritic cells are isolated from a donor supplying a graft to said subject.

68. The method of claim 66, wherein said CD25+ CD4+ T cells are isolated from a donor supplying a graft to said subject.

69. The method of claim 66, wherein said CD25+ CD4+ T cells are syngeneic or allogeneic, with respect to said dendritic cells and said subject.

70. The method of claim 61, wherein said immune response is a result of host versus graft disease.

71. The method of claim 70, wherein said dendritic cells are isolated from said subject.

72. The method of claim 70, wherein said CD25+ CD4+ T cells are isolated from said subject.

73. The method of claim 70, wherein said CD25+ CD4+ T cells are syngeneic or allogeneic, with respect to said dendritic cells.

74. The method of claim 70, wherein said CD25+ CD4+ T cells are CD62L+.

75. The method of claim 70, wherein said antigenic peptide or antigenic protein is derived from said graft.

76.-78. (canceled)

79. The method of claim 76, wherein said dendritic cells are isolated from said subject.

80. The method of claim 76, wherein said dendritic cells express a costimulatory molecule.

81. The method of claim 80, wherein said dendritic cells are enriched for CD86^{high} expression.

82. The method of claim 76, wherein said CD25+ CD4+ T cells are isolated from said subject.

83. The method of claim 76, wherein said CD25+ CD4+ T cells are enriched for CTLA-4^{high} and/or GITR^{high} expression.

84. The method of claim 76, wherein said expanded CD25+ CD4+ T cells are polyclonal.

85. The method of claim 76, wherein said expanded CD25+ CD4+ T cells are monoclonal.

86. The method of claim 76, further comprising the step of adding a cytokine in step (a).

87. A method for delaying onset, reducing incidence, suppressing or treating autoimmunity in a subject, comprising the steps of:
c. Culturing an isolated dendritic cell population with an antigenic peptide or an antigenic protein associated with an autoimmune response in a subject, or a derivative thereof; and
d. Administering the dendritic cells in (a) to a subject, whereby said dendritic cells contact CD25+ CD4+ T cells, resulting in CD25+ CD4+ T cell expansion in said subject,
wherein expanded CD25+ CD4+ T cells suppress an autoimmune response in said subject, thereby delaying onset, reducing incidence, suppressing or treating autoimmunity.

88. The method of claim 87, wherein said dendritic cells are isolated from said subject.

89. The method of claim 87, wherein said dendritic cells express a costimulatory molecule.

90. The method of claim 89, wherein said dendritic cells are enriched for CD86^{high} expression.

91. The method of claim 87, further comprising the step of adding a cytokine in step (b).

92. The method of claim 87, wherein said antigenic peptide or protein is expressed in pancreatic β cells.

93. The method of claim 87, wherein said antigenic peptide is a BDC mimetope.

94. The method of claim 87, wherein said autoimmune response is directed against multiple autoantigens.

95. The method of claim 87, wherein said autoimmune response results in the development of type I diabetes.

96.-97. (canceled)

98. The method of claim 83, wherein dendritic cell contact with said CD25+ CD4+ T cells results in enhanced dendritic cell longevity, antigen persistence, or a combination thereof.

99.-130. (canceled)
